(19) United States
(12) Patent Application Publication
(10) Pub. No.: US 2026/0365622 A1
(43) Pub. Date: Nov. 19, 2026

(54) CRYSTALLINE CARBAZOLE DERIVATIVE AND METHODS OF MAKING THEREOF

(71) Applicant: Raziel Therapeutics Ltd., Rehovot (IL)

(72) Inventors: Sharon COHEN-VERED, Kfar Saba (IL); Eran BLAUGRUND, Rehovot (IL); Racheli GUETA, Rehovot (IL); Alon BLOOMENFELD, Hod Hasharon (IL)

(21) Appl. No.: 19/126,583

(22) PCT Filed: Nov. 7, 2023
(86) PCT No.: PCT/IB2023/061200

§ 371 Date: May 1, 2025

Related U.S. Application Data
(60) Provisional application No. 63/382,608, filed on Nov. 7, 2022.

(51) Int. Cl. C07D 209/88

(52) U.S. Cl. CPC C07D 209/88

(57) ABSTRACT

A crystalline carbazole derivative, pharmaceutical compositions thereof, and methods of treatment are provided herein.

### CROSS-REFERENCE TO RELATED APPLICATIONS

This application claims the benefit of U.S. Provisional Patent Application No. 63/382,608, filed Nov. 7, 2022, which is incorporated herein by reference in its entirety.

### BACKGROUND

Fat-related disorders present many aesthetic and health-related problems. While advances have been made in recent years, there remains a need for new treatments for fat-related disorders.

### SUMMARY

In one aspect, provided herein is anhydrous crystalline 5-(3,6-dibromo-9H-carbazol-9-yl)-N,N,N-trimethylpentan-1-aminium chloride (Compound A):

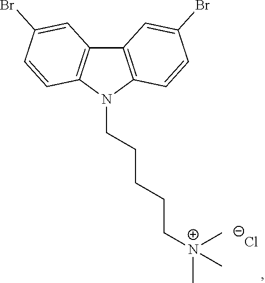 [SMILES: C[N+](C)(C)CCCCCn1c2ccc(Br)cc2c2cc(Br)ccc21 ; [Cl-]]

the anhydrous crystalline 5-(3,6-dibromo-9H-carbazol-9-yl)-N,N,N-trimethylpentan-1-aminium chloride is characterized as having:
(a) an X-ray powder diffraction pattern comprising peaks at 3.0±0.2° 2-θ, 22.3±0.2° 2-θ, and 31.2±0.2° 2-θ, as measured using Cu Kα1 radiation with an X-ray wavelength of 1.54060 Å;
(b) an X-ray powder diffraction pattern substantially the same as shown in FIG. 8; or
(c) combinations thereof.

In one aspect, provided herein is a mixture of anhydrous crystalline forms of 5-(3,6-dibromo-9H-carbazol-9-yl)-N,N,N-trimethylpentan-1-aminium chloride (Compound A):

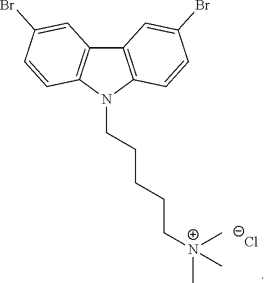 [SMILES: C[N+](C)(C)CCCCCn1c2ccc(Br)cc2c2cc(Br)ccc21 ; [Cl-]]

In some embodiments, the mixture comprises anhydrous crystalline 5-(3,6-dibromo-9H-carbazol-9-yl)-N,N,N-trimethylpentan-1-aminium chloride is characterized as having an X-ray powder diffraction pattern comprising peaks at 3.0±0.2° 2-θ, 4.8±0.2° 2-θ, 22.3±0.2° 2-θ, 24.8±0.2° 2-θ, 26.5±0.2° 2-θ, and 31.2±0.2° 2-θ, as measured using Cu Kα1 radiation with an X-ray wavelength of 1.54060 Å. In some embodiments, the anhydrous crystalline forms of Compound A are formed by a process comprising exposing Compound A to an elevated temperature. In some embodiments, the anhydrous crystalline forms of Compound A are formed by a process comprising drying Compound A to lower water content.

In one aspect, provided herein is a process for the preparation of anhydrous crystalline 5-(3,6-dibromo-9H-carbazol-9-yl)-N,N,N-trimethylpentan-1-aminium chloride (Compound A), the process comprising reducing water content in a sample of Compound A and maintaining the sample at a low relative humidity. In some embodiments, reducing water content comprises heating the sample of Compound A. In some embodiments, the low relative humidity is less than about 30%.

In one aspect, provided herein is a process for the preparation of Form 1 of crystalline 5-(3,6-dibromo-9H-carbazol-9-yl)-N,N,N-trimethylpentan-1-aminium chloride (Compound A Form 1), the process comprising exposing a sample of Compound A to a humid environment. In some embodiments, the sample of Compound A is an unstable crystalline form. In some embodiments, the sample of Compound A is anhydrous.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the X-ray powder diffraction (XRPD) pattern for Form 1 of crystalline Compound A.

FIG. 2 shows the differential scanning calorimetry (DSC) thermogram for Form 1 of crystalline Compound A.

FIG. 3 shows the thermogravimetric analysis (TGA) pattern for Form 1 of crystalline Compound A.

FIG. 4 shows the X-ray powder diffraction (XRPD) pattern for crystalline Form 2 of Compound A.

FIG. 5 shows the differential scanning calorimetry (DSC) thermogram for Form 2 of crystalline Compound A.

FIG. 6 shows the thermogravimetric analysis (TGA) pattern for Form 2 of crystalline Compound A.

FIG. 7 shows the X-ray powder diffraction (XRPD) pattern for Form 3 of crystalline Compound A.

FIG. 8 shows the X-ray powder diffraction (XRPD) pattern for Form 4 of crystalline Compound A.

### DETAILED DESCRIPTION

While small molecule inhibitors are often initially evaluated for their activity when dissolved in solution, solid state characteristics such as polymorphism are also important. Polymorphic forms of a drug substance can have different physical properties, including melting point, apparent solubility, dissolution rate, optical and mechanical properties, vapor pressure, and density. These properties can have a direct effect on the ability to process or manufacture a drug substance and the drug product. Moreover, differences in these properties can and often lead to different pharmacokinetics profiles for different polymorphic forms of a drug. Therefore, polymorphism is often an important factor under regulatory review of the ‘sameness’ of drug products from various manufacturers. For example, polymorphism has been evaluated in many multi-million dollar and even multi-billion dollar drugs, such as warfarin sodium, famotidine, and ranitidine. Polymorphism can affect the quality, safety, and/or efficacy of a drug product. Thus, there still remains a need for polymorphs of drug products. The present disclosure addresses this need and provides related advantages as well.

### Compound A

As used herein, Compound A refers to 5-(3,6-dibromo-9H-carbazol-9-yl)-N,N,N-trimethylpentan-1-aminium chloride, which has the chemical structure shown below:

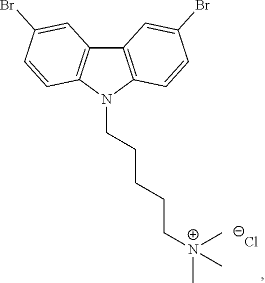 [SMILES: C[N+](C)(C)CCCCCn1c2ccc(Br)cc2c2cc(Br)ccc21 ; [Cl-]]

Compound A, namely 5-(3,6-dibromo-9H-carbazol-9-yl)-N,N,N-trimethylpentan-1-aminium chloride, has been prepared previously (see, WO 2013/072915 and U.S. Pat. No. 9,447,040).

In some embodiments disclosed herein, Compound A is amorphous. In some embodiments disclosed herein, Compound A is crystalline.

### I. CRYSTALLINE FORM OF COMPOUND A

Compound A exhibits polymorphism and the crystalline forms of Compound A that are made according to the methods described herein may be characterized by any methodology known in the art. For example, the crystalline Compound A is characterized by X-ray powder diffraction (XRPD), differential scanning calorimetry (DSC), thermogravimetric analysis (TGA), hot-stage microscopy, as well as other analysis methods such as Raman spectroscopy, solid state nuclear magnetic resonance (ssNMR) spectroscopy, and infrared (IR) spectroscopy). In some embodiments, crystallinity of a solid form is determined by X-Ray Powder Diffraction (XRPD).

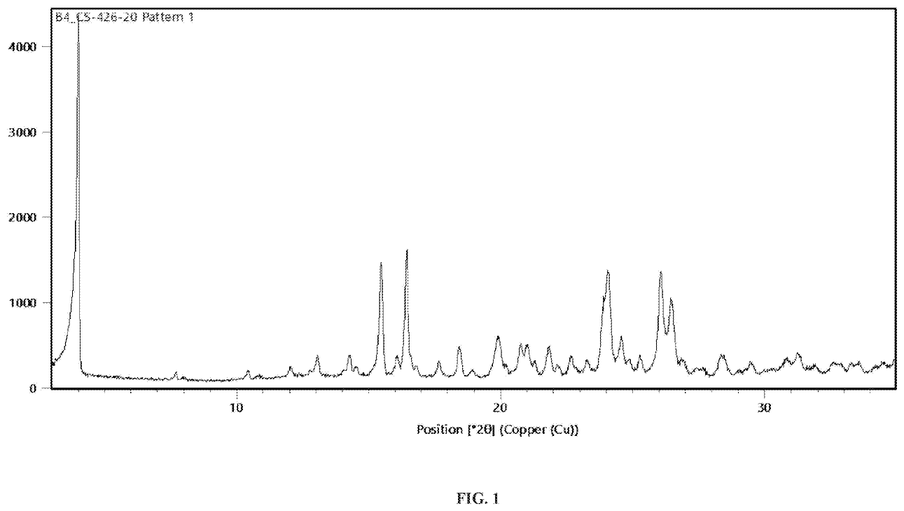

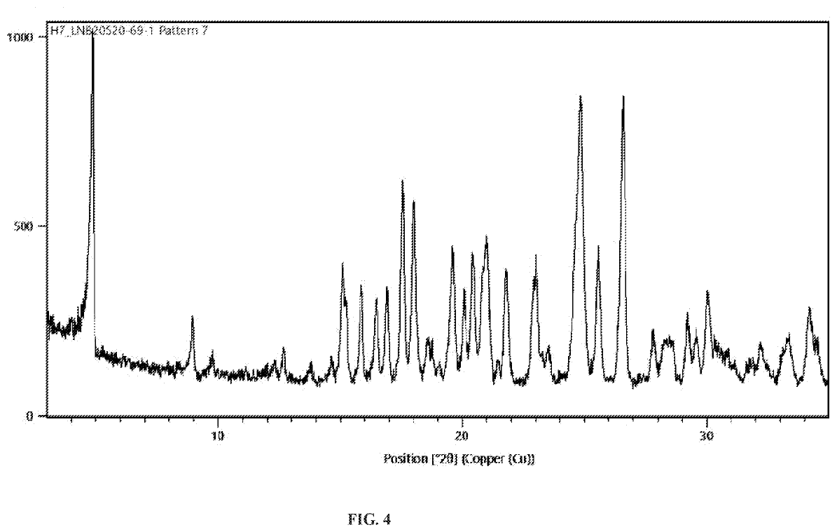

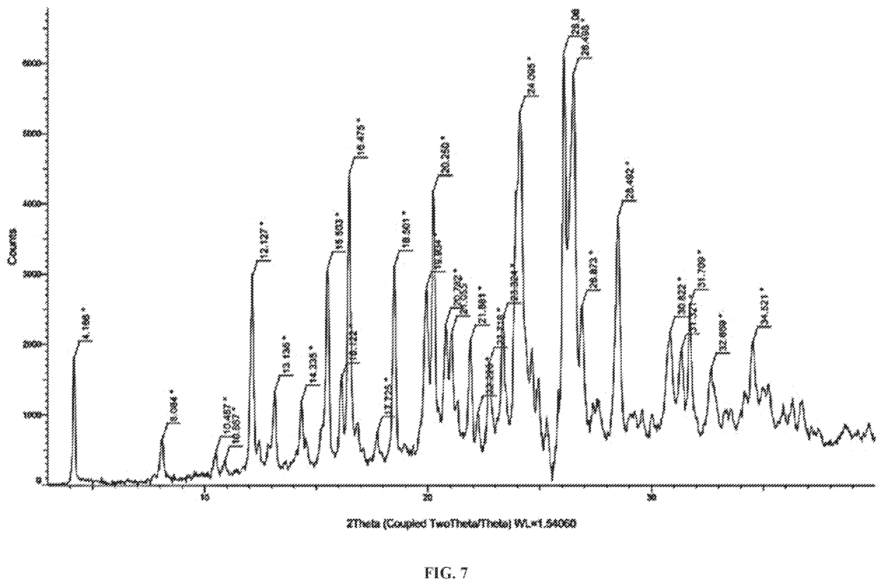

XRPD: A crystalline form of Compound A according to the invention may be characterized by XRPD such as shown in FIG. 1, FIG. 4, or FIG. 7. The relative intensities of XRPD peaks can vary, depending upon the particle size, the sample preparation technique, the sample mounting procedure and the particular instrument employed. Moreover, instrument variation and other factors can affect the 2-θ values. Therefore, the XRPD peak assignments can vary, for example by plus or minus about 0.2 degrees.

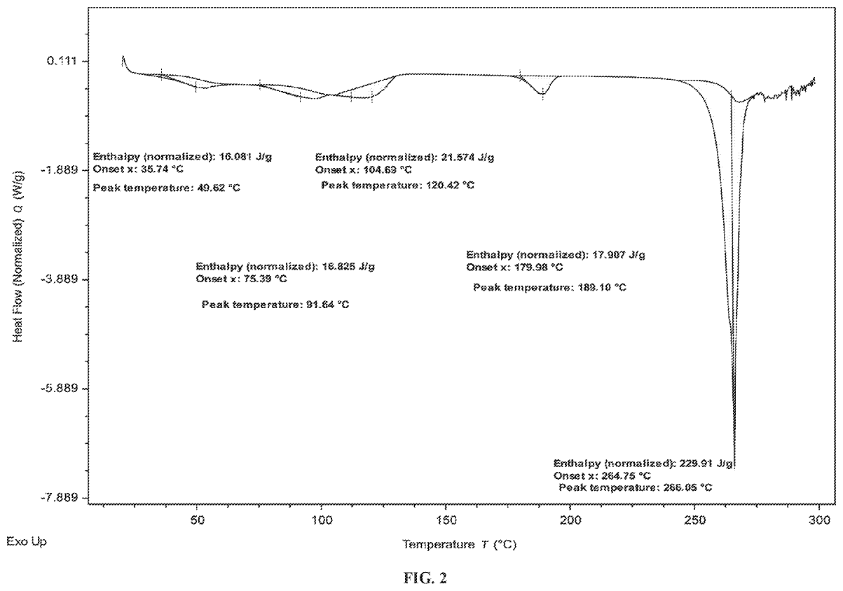

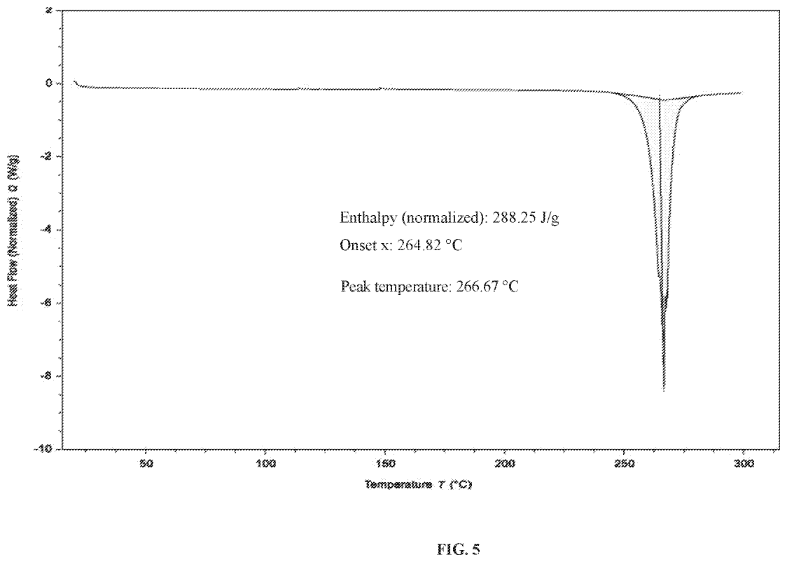

DSC: A crystalline form of Compound A according to the invention can also be identified by its characteristic DSC thermograms such as shown in FIG. 2 or FIG. 5. For DSC, it is known that the temperatures observed will depend upon the rate of temperature change as well as sample preparation technique and the particular instrument employed. Thus, the values reported herein relating to DSC thermograms can vary, for example by plus or minus about 4 °C.

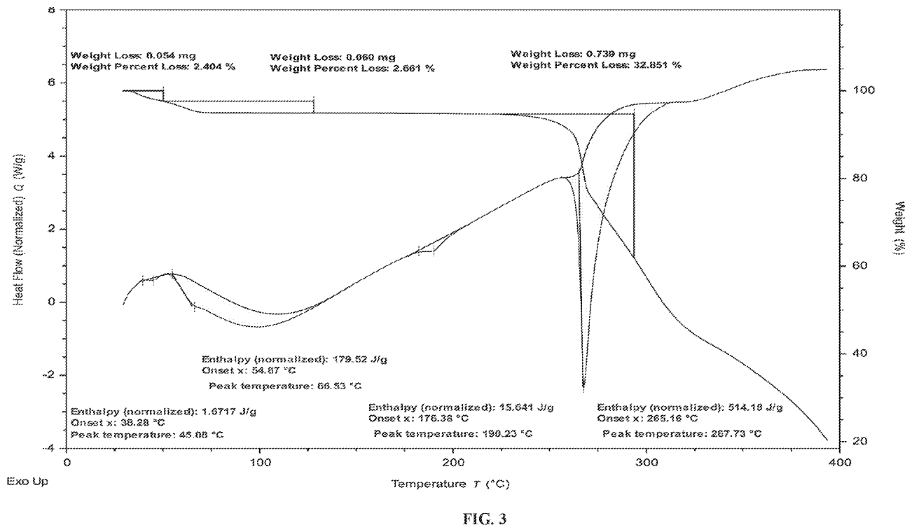

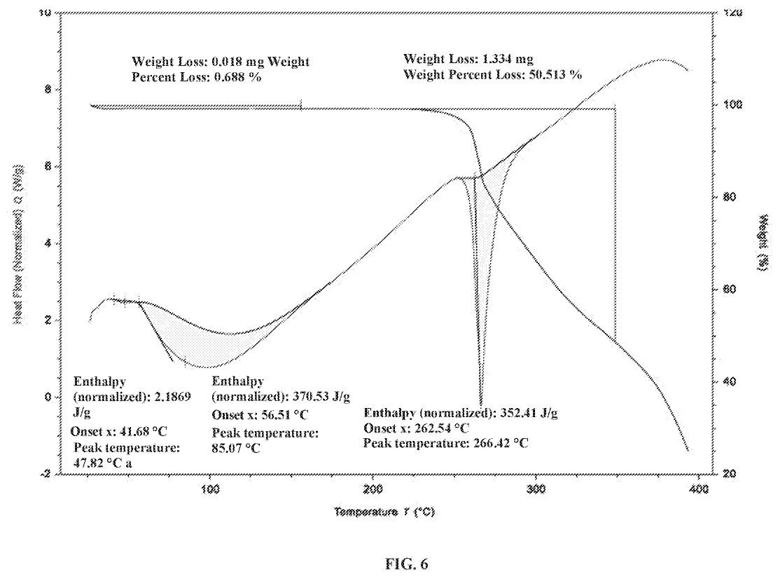

TGA: A crystalline form of Compound A form of the invention may also give rise to thermal behavior different from that of the amorphous material or another polymorphic form. Thermal behavior may be measured in the laboratory by thermogravimetric analysis (TGA) which may be used to distinguish some polymorphic forms from others. In one aspect, the crystalline form of Compound A may be characterized by thermogravimetric analysis. Crystalline Compound A can be identified by its characteristic TGA thermograms such as shown in FIG. 3 or FIG. 6.

The crystalline form of Compound A is useful in the production of medicinal preparations and can be obtained by means of a crystallization process. Semi-crystalline forms can also be obtained. In some embodiments, semi-crystalline forms of Compound A comprise 1 or more of the crystalline forms described herein and/or amorphous Compound A. In some embodiments, a solidification process is used to obtain the amorphous form. In various embodiments, the crystallization is carried out by either generating the desired compound (for example Compound A) in a reaction mixture and isolating the desired crystalline form from the reaction mixture, or by dissolving raw compound in a solvent, optionally with heat, followed by crystallizing/solidifying the product by cooling (including active cooling) and/or by the addition of an antisolvent for a period of time. The crystallization or solidification may be followed by drying carried out under controlled conditions until the desired water content is reached for the desired crystalline form.

In various embodiments, the crystalline Compound A disclosed herein is stable at room temperature. In some examples, the crystalline Compound A is stable and can be stored at mom temperature for an extended period of time without chemical degradation or change in the crystalline form. In some examples, the crystalline Compound A is stable and can be stored at room temperature for an extended period of time without significant chemical degradation or change in the crystalline form. In some examples, the crystalline Compound A is stable and can be stored at room temperature for a time period of at least about 1 day, 2 days, 3 days, 4 days, 5 days, 6 days, or 7 days. In some examples, the crystalline Compound A is stable and can be stored at room temperature for a time period of more than about 7 days. In some examples, the crystalline Compound A is stable and can be stored at room temperature for a time period of 10-20 days. In some examples, the crystalline Compound A is stable and can be stored at room temperature for a time period of 10-15 days. In some examples, the crystalline Compound A is stable and can be stored at room temperature for a time period of 15-20 days. In some examples, the crystalline Compound A is stable and can be stored at room temperature for a time period of 10-12 days. In some examples, the crystalline Compound A is stable and can be stored at room temperature for a time period of 12-14 days. In some examples, the crystalline Compound A is stable and can be stored at room temperature for a time period of 14-16 days. In some examples, the crystalline Compound A is stable and can be stored at room temperature for a time period of 16-18 days. In some examples, the crystalline Compound A is stable and can be stored at room temperature for a time period of 18-20 days. In some examples, the crystalline Compound A is stable and can be stored at room temperature for a time period of 1-2 days, 1-3 days, 1-4 days, 1-5 days, 1-6 days, 1-7 days, 2-3 days, 2-4 days, 2-5 days, 2-6 days, 2-7 days, 3-4 days, 3-5 days, 3-6 days, 3-7 days, 4-5 days, 4-6 days, 4-7 days, 5-6 days, 5-7 days, or 6-7 days. In some examples, the crystalline Compound A can be stored at room temperature for a time period of at least 1 day, 2 days, 3 days, 4 days, 5 days, 6 days, or 7 days. In some embodiments, the crystalline Compound A is stable and can be stored at room temperature for a period of time of at least 1 month, at least 2 months, at least 3 months, at least 4 months, at least 5 months, at least 6 months, at least 7 months, at least 8 months, at least 9 months, at least 10 months, at least 11 months, or at least 12 months. In some embodiments, the crystalline Compound A is stable and can be stored at room temperature for over 1 year without chemical degradation or change in the crystalline form. In some embodiments, the crystalline Compound A is stable and can be stored at room temperature for over 1 year without significant chemical degradation or change in the crystalline form. In some embodiments, the crystalline Compound A is stable and can be stored at room temperature for a period of time of at least 12 months, at least 14 months, at least 16 months, at least 18 months, at least 20 months, at least 22 months, or at least 24 months. In some embodiments, the crystalline Compound A is stable and can be stored at room temperature between 1 to 2 years without chemical degradation or change in the crystalline form. In some embodiments, the crystalline Compound A is stable and can be stored at room temperature between 1 to 2 years without significant chemical degradation or change in the crystalline form. In some embodiments, the crystalline Compound A is stable and can be stored at room temperature for over 2 years. In some embodiments, the crystalline Compound A is stable and can be stored at room temperature for a period of time of at least 24 months, at least 30 months, or at least 36 months. In some embodiments, crystalline Compound A is stable and can be stored at room temperature for 36 months. In some embodiments, the crystalline Compound A is stable and can be stored at room temperature for a period of time of 12-18 months, 14-20 months, 16-22 months, 18-24 months, 20-26 months, 22-38 months, or 24-40 months.

In various embodiments, the crystalline Compound A disclosed herein is stable at temperatures above the room temperature and/or at high relative humidity (RH). In some examples, the crystalline Compound A is stable and can be stored at about 40 °C at about 75% RH for an extended period of time without chemical degradation or change in the crystalline form. In some examples, the crystalline Compound A is stable and can be stored at about 40 °C at about 75% RH for an extended period of time without significant chemical degradation or change in the crystalline form. In some examples, the crystalline Compound A is stable and can be stored at about 40 °C and at about 75% RH for a time period of at least about 1 day, 2 days, 3 days, 4 days, 5 days, 6 days, or 7 days. In some examples, the crystalline Compound A is stable and can be stored at about 40 °C and at about 75% RH for a time period of more than about 7 days. In some examples, the crystalline Compound A is stable and can be stored at about 40 °C and at about 75% RH for a time period of 1-2 days, 1-3 days, 1-4 days, 1-5 days, 1-6 days, 1-7 days, 2-3 days, 2-4 days, 2-5 days, 2-6 days, 2-7 days, 3-4 days, 3-5 days, 3-6 days, 3-7 days, 4-5 days, 4-6 days, 4-7 days, 5-6 days, 5-7 days, or 6-7 days. In some examples, the crystalline Compound A is stable and can be stored at about 40 °C and at about 75% RH for a time period of at least 1 day, 2 days, 3 days, 4 days, 5 days, 6 days, or 7 days.

In various embodiments, the crystalline Compound A disclosed herein is stable at elevated temperature. In some examples, the crystalline Compound A is stable and can be stored at about 80 °C for an extended period of time without chemical degradation or change in the crystalline form. In some examples, the crystalline Compound A is stable and can be stored at about 80 °C for an extended period of time without significant chemical degradation or change in the crystalline form. In some examples, the crystalline Compound A is stable and can be stored at about 80 °C for a time period of at least about 1 day, 2 days, 3 days, 4 days, 5 days, 6 days, or 7 days. In some examples, the crystalline Compound A is stable and can be stored at about 80 °C for a time period of more than about 7 days. In some examples, the crystalline Compound A can be stored at about 80 °C for a time period of 1-2 days, 1-3 days, 1-4 days, 1-5 days, 1-6 days, 1-7 days, 2-3 days, 2-4 days, 2-5 days, 2-6 days, 2-7 days, 3-4 days, 3-5 days, 3-6 days, 3-7 days, 4-5 days, 4-6 days, 4-7 days, 5-6 days, 5-7 days, or 6-7 days. In some examples, the crystalline Compound A is stable and can be stored at about 80 °C for a time period of at least 1 day, 2 days, 3 days, 4 days, 5 days, 6 days, or 7 days.

### Crystalline Compound A Form 1

FIG. 1 shows the X-ray powder diffraction (XRPD) pattern for Form 1 of crystalline Compound A.

FIG. 2 shows the differential scanning calorimetry (DSC) thermogram for Form 1 of crystalline Compound A.

FIG. 3 shows the thermogravimetric analysis (TGA) pattern for Form 1 of crystalline Compound A.

In one aspect, provided herein is crystalline 5-(3,6-dibromo-9H-carbazol-9-yl)-N,N,N-trimethylpentan-1-aminium chloride (Compound A). Some embodiments provide a composition comprising crystalline 5-(3,6-dibromo-9H-carbazol-9-yl)-N,N,N-trimethylpentan-1-aminium chloride. In some embodiments, crystalline 5-(3,6-dibromo-9H-carbazol-9-yl)-N,N,N-trimethylpentan-1-aminium chloride Form 1 (Compound A Form 1) is characterized as having:
(a) an X-ray powder diffraction pattern comprising peaks at 4.0±0.2° 2-θ, 16.5±0.2° 2-θ, and 15.5±0.2° 2-θ, as measured using Cu Kα1 radiation with an X-ray wavelength of 1.54060 Å;
(b) an X-ray powder diffraction pattern substantially the same as shown in FIG. 1, as measured using Cu Kα1 radiation with an X-ray wavelength of 1.54060 Å;
(c) a differential scanning calorimetry (DSC) thermogram comprising:
   i) an endotherm in the range of about 260-270 °C; and
   ii) an endotherm in the range of about 175-195 °C;
(d) a differential scanning calorimetry (DSC) thermogram comprising:
   i) an endotherm with an onset of about 265 °C and a peak of about 266 °C; and
   ii) an endotherm with an onset of about 180 °C and a peak of about 190 °C;
(e) a differential scanning calorimetry (DSC) thermogram substantially the same as shown in FIG. 2;
(f) a Thermogravimetric Analysis (TGA) thermogram substantially the same as shown in FIG. 3;
(g) an unchanged XRPD after storage at 40 °C and 75% relative humidity (RH) for 7 days;
(h) an unchanged XRPD after storage at 80 °C for 7 days;
or
(i) combinations thereof.

In some embodiments, Form 1 of crystalline Compound A is characterized as having an X-ray powder diffraction pattern substantially the same as shown in FIG. 1 as measured using Cu Kα1 radiation with an X-ray wavelength of 1.54060 Å.

In some embodiments, Form 1 of crystalline Compound A is characterized as having an X-ray powder diffraction pattern comprising peaks at 4.0±0.2° 2-θ, 16.5±0.2° 2-θ, and 15.5±0.2° 2-θ, as measured using Cu Kα1 radiation with an X-ray wavelength of 1.54060 Å. In some embodiments, Form 1 of crystalline Compound A is characterized as having an X-ray powder diffraction pattern comprising peaks at 4.0±0.1° 2-θ, 16.5±0.1° 2-θ, and 15.5±0.1° 2-θ, as measured using Cu Kα1 radiation with an X-ray wavelength of 1.54060 Å. In some embodiments, Form 1 of crystalline Compound A is characterized as having an X-ray powder diffraction pattern comprising peaks at about 4.0° 2-θ, about 16.5° 2-θ, and about 15.5° 2-θ, as measured using Cu Kα1 radiation with an X-ray wavelength of 1.54060 Å.

In some embodiments, the X-ray powder diffraction pattern of Form 1 of crystalline Compound A further comprises at least one peak selected from 24.1±0.2° 2-θ, 26.0±0.2° 2-θ, and 26.1±0.2° 2-θ, as measured using Cu Kα1 radiation with an X-ray wavelength of 1.54060 Å. In some embodiments, the X-ray powder diffraction pattern of Form 1 of crystalline Compound A further comprises at least one peak selected from 24.1±0.1° 2-θ, 26.0±0.1° 2-θ, and 26.1±0.1° 2-θ, as measured using Cu Kα1 radiation with an X-ray wavelength of 1.54060 Å. In some embodiments, the X-ray powder diffraction pattern of Form 1 of crystalline Compound A further comprises at least one peak selected from about 24.1° 2-θ, about 26.0° 2-θ, and about 26.1° 2-θ, as measured using Cu Kα1 radiation with an X-ray wavelength of 1.54060 Å.

In some embodiments, the X-ray powder diffraction pattern of Form 1 of crystalline Compound A further comprises at least one peak selected from 19.9±0.2° 2-θ, 23.8±0.2° 2-θ, and 26.5±0.2° 2-θ, as measured using Cu Kα1 radiation with an X-ray wavelength of 1.54060 Å. In some embodiments, the X-ray powder diffraction pattern of Form 1 of crystalline Compound A further comprises at least one peak selected from 19.9±0.1° 2-θ, 23.8±0.1° 2-θ, and 26.5±0.1° 2-θ, as measured using Cu Kα1 radiation with an X-ray wavelength of 1.54060 Å. In some embodiments, the X-ray powder diffraction pattern of Form 1 of crystalline Compound A further comprises at least one peak selected from about 19.9° 2-θ, about 23.8° 2-θ, and about 26.5° 2-θ, as measured using Cu Kα1 radiation with an X-ray wavelength of 1.54060 Å.

In some embodiments, the X-ray powder diffraction pattern of Form 1 of crystalline Compound A comprises at least one peak selected from 4.0±0.2° 2-θ, 15.5±0.2° 2-θ, 16.5±0.2° 2-θ, 19.9±0.2° 2-θ, 23.8±0.2° 2-θ, 24.1±0.2° 2-θ, 26.0±0.2° 2-θ, 26.1±0.2° 2-θ, and 26.5±0.2° 2-θ, as measured using Cu Kα1 radiation with an X-ray wavelength of 1.54060 Å. In some embodiments, the X-ray powder diffraction pattern of Form 1 of crystalline Compound A comprises at least two peaks selected from 4.0±0.2° 2-θ, 15.5±0.2° 2-θ, 16.5±0.2° 2-θ, 19.9±0.2° 2-θ, 23.8±0.2° 2-θ, 24.1±0.2° 2-θ, 26.0±0.2° 2-θ, 26.1±0.2° 2-θ, and 26.5±0.2° 2-θ, as measured using Cu Kα1 radiation with an X-ray wavelength of 1.54060 Å. In some embodiments, the X-ray powder diffraction pattern of Form 1 of crystalline Compound A comprises at least three peaks selected from 4.0±0.2° 2-θ, 15.5±0.2° 2-θ, 16.5±0.2° 2-θ, 19.9±0.2° 2-θ, 23.8±0.2° 2-θ, 24.1±0.2° 2-θ, 26.0±0.2° 2-θ, 26.1±0.2° 2-θ, and 26.5±0.2° 2-θ, as measured using Cu Kα1 radiation with an X-ray wavelength of 1.54060 Å. In some embodiments, the X-ray powder diffraction pattern of Form 1 of crystalline Compound A comprises at least four peaks selected from 4.0±0.2° 2-θ, 15.5±0.2° 2-θ, 16.5±0.2° 2-θ, 19.9±0.2° 2-θ, 23.8±0.2° 2-θ, 24.1±0.2° 2-θ, 26.0±0.2° 2-θ, 26.1±0.2° 2-θ, and 26.5±0.2° 2-θ, as measured using Cu Kα1 radiation with an X-ray wavelength of 1.54060 Å. In some embodiments, the X-ray powder diffraction pattern of Form 1 of crystalline Compound A comprises at least five peaks selected from 4.0±0.2° 2-θ, 15.5±0.2° 2-θ, 16.5±0.2° 2-θ, 19.9±0.2° 2-θ, 23.8±0.2° 2-θ, 24.1±0.2° 2-θ, 26.0±0.2° 2-θ, 26.1±0.2° 2-θ, and 26.5±0.2° 2-θ, as measured using Cu Kα1 radiation with an X-ray wavelength of 1.54060 Å. In some embodiments, the X-ray powder diffraction pattern of Form 1 of crystalline Compound A comprises at least six peaks selected from 4.0±0.2° 2-θ, 15.5±0.2° 2-θ, 16.5±0.2° 2-θ, 19.9±0.2° 2-θ, 23.8±0.2° 2-θ, 24.1±0.2° 2-θ, 26.0±0.2° 2-θ, 26.1±0.2° 2-θ, and 26.5±0.2° 2-θ, as measured using Cu Kα1 radiation with an X-ray wavelength of 1.54060 Å. In some embodiments, the X-ray powder diffraction pattern of Form 1 of crystalline Compound A comprises at least seven peaks selected from 4.0±0.2° 2-θ, 15.5±0.2° 2-θ, 16.5±0.2° 2-θ, 19.9±0.2° 2-θ, 23.8±0.2° 2-θ, 24.1±0.2° 2-θ, 26.0±0.2° 2-θ, 26.1±0.2° 2-θ, and 26.5±0.2° 2-θ, as measured using Cu Kα1 radiation with an X-ray wavelength of 1.54060 Å. In some embodiments, the X-ray powder diffraction pattern of Form 1 of crystalline Compound A comprises at least eight peaks selected from 4.0±0.2° 2-θ, 15.5±0.2° 2-θ, 16.5±0.2° 2-θ, 19.9±0.2° 2-θ, 23.8±0.2° 2-θ, 24.1±0.2° 2-θ, 26.0±0.2° 2-θ, 26.1±0.2° 2-θ, and 26.5±0.2° 2-θ, as measured using Cu Kα1 radiation with an X-ray wavelength of 1.54060 Å. In some embodiments, the X-ray powder diffraction pattern of Form 1 of crystalline Compound A comprises peaks at 4.0±0.2° 2-θ, 15.5±0.2° 2-θ, 16.5±0.2° 2-θ, 19.9±0.2° 2-θ, 23.8±0.2° 2-θ, 24.1±0.2° 2-θ, 26.0±0.2° 2-θ, 26.1±0.2° 2-θ, and 26.5±0.2° 2-θ, as measured using Cu Kα1 radiation with an X-ray wavelength of 1.54060 Å. In some embodiments, the X-ray powder diffraction pattern of Form 1 of crystalline Compound A comprises peaks at 4.0±0.1° 2-θ, 15.5±0.1° 2-θ, 16.5±0.1° 2-θ, 19.9±0.1° 2-θ, 23.8±0.1° 2-θ, 24.1±0.1° 2-θ, 26.0±0.1° 2-θ, 26.1±0.1° 2-θ, and 26.5±0.1° 2-θ, as measured using Cu Kα1 radiation with an X-ray wavelength of 1.54060 Å. In some embodiments, the X-ray powder diffraction pattern of Form 1 of crystalline Compound A comprises peaks at about 4.0° 2-θ, about 15.5° 2-θ, about 16.5° 2-θ, about 19.9° 2-θ, about 23.8° 2-θ, about 24.1° 2-θ, about 26.0° 2-θ, about 26.1° 2-θ, and about 26.5° 2-θ, as measured using Cu Kα1 radiation with an X-ray wavelength of 1.54060 Å.

In some embodiments, Form 1 of crystalline Compound A is characterized by a differential scanning calorimetry (DSC) thermogram substantially the same as shown in FIG. 2. In some embodiments. Form 1 of crystalline Compound A is characterized by a differential scanning calorimetry (DSC) thermogram comprising: an endotherm in the range of about 260-270 °C; and an endotherm in the range of about 175-195 °C. In some embodiments, Form 1 of crystalline Compound A is characterized by a differential scanning calorimetry (DSC) thermogram comprising: an endotherm with an onset of about 265 °C and a peak of about 266 °C; and an endotherm with an onset of about 180 °C and a peak of about 190 °C.

In some embodiments, Form 1 of crystalline Compound A is characterized by an endotherm in the range of about 260-270 °C, for example at about 260-270 °C, 260-268 °C, 260-266 °C, 260-264 °C, 260-262 °C, 262-270 °C, 262-268 °C, 262-266 °C, 262-264 °C, 264-270 °C, 264-268 °C, 264-266 °C, 266-270 °C, 266-268 °C, or 26-270 °C in the DSC thermogram. In some examples. Form 1 of crystalline Compound A is characterized by an endotherm at about 266 °C in the DSC thermogram.

In some embodiments, Form 1 of crystalline Compound A is further characterized by an endotherm in the range of about 175-195 °C, for example at about 175-195 °C, 175-190 °C, 175-185 °C, 175-180 °C, 180-195 °C, 180-190 °C, 180-185 °C, 185-195 °C, 185-190 °C, or 190-195 °C in the DSC thermogram. In some examples, Form 1 of crystalline Compound A is further characterized by an endotherm at about 190 °C in the DSC thermogram.

In some embodiments, Form 1 of crystalline Compound A is characterized by a Thermogravimetric Analysis (TGA) thermogram substantially the same as shown in FIG. 3. In various embodiments, Form 1 of crystalline Compound A decomposes above a temperature of about 150 °C, about 200 °C, about 250 °C, about 300 °C, about 350 °C, about 400 °C, about 450 °C, about 500 °C, about 550 °C or above 600 °C. In some examples, crystalline Compound A Form 1 decomposes above a temperature of about 250 °C.

### Crystalline Compound A Form 2

FIG. 4 shows the X-ray powder diffraction (XRPD) pattern for Form 2 of crystalline Compound A.

FIG. 5 shows the differential scanning calorimetry (DSC) thermogram for Form 2 of crystalline Compound A

FIG. 6 shows the thermogravimetric analysis (TGA) pattern for Form 2 of crystalline Compound A.

In one aspect, provided herein is crystalline 5-(3,6-dibromo-9H-carbazol-9-yl)-N,N,N-trimethylpentan-1-aminium chloride (Compound A). Some embodiments provide a composition comprising crystalline 5-(3,6-dibromo-9H-carbazol-9-yl)-N,N,N-trimethylpentan-1-aminium chloride. In some embodiments, crystalline 5-(3,6-dibromo-9H-carbazol-9-yl)-N,N,N-trimethylpentan-1-aminium chloride Form 2 (Compound A Form 2) is characterized as having:
(a) an X-ray powder diffraction pattern comprising peaks at 4.8±0.2° 2-θ, 26.5±0.2° 2-θ, and 24.8±0.2° 2-θ, as measured using Cu Kα1 radiation with an X-ray wavelength of 1.54060 Å;
(b) an X-ray powder diffraction pattern substantially the same as shown in FIG. 4 as measured using Cu Kα1 radiation with an X-ray wavelength of 1.54060 Å;
(c) a differential scanning calorimetry (DSC) thermogram comprising:
   i) an endotherm in the range of about 260-270 °C;
(d) a differential scanning calorimetry (DSC) thermogram comprising:
   i) an endotherm with an onset of about 265 °C and a peak of about 266 °C:
(e) a differential scanning calorimetry (DSC) thermogram substantially the same as shown in FIG. 5;
(f) a Thermogravimetric Analysis (TGA) thermogram substantially the same as shown in FIG. 6;
(g) an unchanged XRPD after storage at 80 °C for 7 days;
or
(h) combinations thereof.

In some embodiments, Form 2 of crystalline Compound A is characterized as having an X-ray powder diffraction pattern substantially the same as shown in FIG. 4.

In some embodiments, Form 2 of crystalline Compound A is characterized as having an X-ray powder diffraction pattern comprising peaks at 4.8±0.2° 2-θ, 26.5±0.2° 2-θ, and 24.8±0.2° 2-θ, as measured using Cu Kα1 radiation with an X-ray wavelength of 1.54060 Å. In some embodiments, Form 2 of crystalline Compound A is characterized as having an X-ray powder diffraction pattern comprising peaks at 4.8±0.1° 2-θ, 26.5±0.1° 2-θ, and 24.8±0.1° 2-θ, as measured using Cu Kai radiation with an X-ray wavelength of 1.54060 Å. In some embodiments, Form 2 of crystalline Compound A is characterized as having an X-ray powder diffraction pattern comprising peaks at about 4.8° 2-θ, about 26.5° 2-θ, and about 24.8° 2-θ, as measured using Cu Kα1 radiation with an X-ray wavelength of 1.54060 Å.

In some embodiments, the X-ray powder diffraction pattern of Form 2 of crystalline Compound A further comprises at least one peak selected from 17.5±0.2° 2-θ, 17.9±0.2° 2-θ, and 21.0±0.2° 2-θ, as measured using Cu Kα1 radiation with an X-ray wavelength of 1.54060 Å. In some embodiments, the X-ray powder diffraction pattern of Form 2 of crystalline Compound A further comprises at least one peak selected from 17.5±0.1° 2-θ, 17.9±0.1° 2-θ, and 21.0±0.1° 2-θ, as measured using Cu Kα1 radiation with an X-ray wavelength of 1.54060 Å. In some embodiments, the X-ray powder diffraction pattern of Form 2 of crystalline Compound A further comprises at least one peak selected from about 17.5° 2-θ, about 17.9° 2-θ, and about 21.0° 2-θ, as measured using Cu Kα1 radiation with an X-ray wavelength of 1.54060 Å.

In some embodiments, the X-ray powder diffraction pattern of Form 2 of crystalline Compound A further comprises at least one peak selected from 19.6±0.2° 2-θ, 25.5±0.2° 2-θ, and 20.4±0.2° 2-θ, as measured using Cu Kα1 radiation with an X-ray wavelength of 1.54060 Å. In some embodiments, the X-ray powder diffraction pattern of Form 2 of crystalline Compound A further comprises at least one peak selected from 19.6±0.1° 2-θ, 25.5±0.1° 2-θ, and 20.4±0.1° 2-θ, as measured using Cu Kα1 radiation with an X-ray wavelength of 1.54060 Å. In some embodiments, the X-ray powder diffraction pattern of Form 2 of crystalline Compound A further comprises at least one peak selected from about 19.6° 2-θ, about 25.5° 2-θ, and about 20.4° 2-θ, as measured using Cu Kα1 radiation with an X-ray wavelength of 1.54060 Å.

In some embodiments, the X-ray powder diffraction pattern of Form 2 of crystalline Compound A comprises at least one peak selected from 4.8±0.2° 2-θ, 17.5±0.2° 2-θ, 17.9±0.2° 2-θ, 19.6±0.2° 2-θ, 20.4±0.2° 2-θ, 21.0±0.2° 2-θ, 24.8±0.2° 2-θ, 25.5±0.2° 2-θ, and 26.5±0.2° 2-θ, as measured using Cu Kα1 radiation with an X-ray wavelength of 1.54060 Å. In some embodiments, the X-ray powder diffraction pattern of Form 2 of crystalline Compound A comprises at least two peaks selected from 4.8±0.2° 2-θ, 17.5±0.2° 2-θ, 17.9±0.2° 2-θ, 19.6±0.2° 2-θ, 20.4±0.2° 2-θ, 21.0±0.2° 2-θ, 24.8±0.2° 2-θ, 25.5±0.2° 2-θ, and 26.5±0.2° 2-θ, as measured using Cu Kα1 radiation with an X-ray wavelength of 1.54060 Å. In some embodiments, the X-ray powder diffraction pattern of Form 2 of crystalline Compound A comprises at least three peaks selected from 4.8±0.2° 2-θ, 17.5±0.2° 2-θ, 17.9±0.2° 2-θ, 19.6±0.2° 2-θ, 20.4±0.2° 2-θ, 21.0±0.2° 2-θ, 24.8±0.2° 2-θ, 25.5±0.2° 2-θ, and 26.5±0.2° 2-θ, as measured using Cu Kα1 radiation with an X-ray wavelength of 1.54060 Å. In some embodiments, the X-ray powder diffraction pattern of Form 2 of crystalline Compound A comprises at least four peaks selected from 4.8±0.2° 2-θ, 17.5±0.2° 2-θ, 17.9±0.2° 2-θ, 19.6±0.2° 2-θ, 20.4±0.2° 2-θ, 21.0±0.2° 2-θ, 24.8±0.2° 2-θ, 25.5±0.2° 2-θ, and 26.5±0.2° 2-θ, as measured using Cu Kα1 radiation with an X-ray wavelength of 1.54060 Å. In some embodiments, the X-ray powder diffraction pattern of Form 2 of crystalline Compound A comprises at least five peaks selected from 4.8±0.2° 2-θ, 17.5±0.2° 2-θ, 17.9±0.2° 2-θ, 19.6±0.2° 2-θ, 20.4±0.2° 2-θ, 21.0±0.2° 2-θ, 24.8±0.2° 2-θ, 25.5±0.2° 2-θ, and 26.5±0.2° 2-θ, as measured using Cu Kα1 radiation with an X-ray wavelength of 1.54060 Å. In some embodiments, the X-ray powder diffraction pattern of Form 2 of crystalline Compound A comprises at least six peaks selected from 4.8±0.2° 2-θ, 17.5±0.2° 2-θ, 17.9±0.2° 2-θ, 19.6±0.2° 2-θ, 20.4±0.2° 2-θ, 21.0±0.2° 2-θ, 24.8±0.2° 2-θ, 25.5±0.2° 2-θ, and 26.5±0.2° 2-θ, as measured using Cu Kα1 radiation with an X-ray wavelength of 1.54060 Å. In some embodiments, the X-ray powder diffraction pattern of Form 2 of crystalline Compound A comprises at least seven peaks selected from 4.8±0.2° 2-θ, 17.5±0.2° 2-θ, 17.9±0.2° 2-θ, 19.6±0.2° 2-θ, 20.4±0.2° 2-θ, 21.0±0.2° 2-θ, 24.8±0.2° 2-θ, 25.5±0.2° 2-θ, and 26.5±0.2° 2-θ, as measured using Cu Kα1 radiation with an X-ray wavelength of 1.54060 Å. In some embodiments, the X-ray powder diffraction pattern of Form 2 of crystalline Compound A comprises at least eight peaks selected from 4.8±0.2° 2-θ, 17.5±0.2° 2-θ, 17.9±0.2° 2-θ, 19.6±0.2° 2-θ, 20.4±0.2° 2-θ, 21.0±0.2° 2-θ, 24.8±0.2° 2-θ, 25.5±0.2° 2-θ, and 26.5±0.2° 2-θ, as measured using Cu Kα1 radiation with an X-ray wavelength of 1.54060 Å. In some embodiments, the X-ray powder diffraction pattern of Form 2 of crystalline Compound A comprises peaks at 4.8±0.2° 2-θ, 17.5±0.2° 2-θ, 17.9±0.2° 2-θ, 19.6±0.2° 2-θ, 20.4±0.2° 2-θ, 21.0±0.2° 2-θ, 24.8±0.2° 2-θ, 25.5±0.2° 2-θ, and 26.5±0.2° 2-θ, as measured using Cu Kα1 radiation with an X-ray wavelength of 1.54060 Å. In some embodiments, the X-ray powder diffraction pattern of Form 2 of crystalline Compound A comprises peaks at 4.8±0.1° 2-θ, 17.5±0.1° 2-θ, 17.9±0.1° 2-θ, 19.6±0.1° 2-θ, 20.4±0.1° 2-θ, 21.0±0.1° 2-θ, 24.8±0.1° 2-θ, 25.5±0.1° 2-θ, and 26.5±0.1° 2-θ, as measured using Cu Kα1 radiation with an X-ray wavelength of 1.54060 Å. In some embodiments, the X-ray powder diffraction pattern of Form 2 of crystalline Compound A comprises peaks at about 4.8° 2-θ, about 17.5° 2-θ, about 17.9° 2-θ, about 19.6° 2-θ, about 20.4° 2-θ, about 21.0° 2-θ, about 24.8° 2-θ, about 25.5° 2-θ, and about 26.6° 2-θ, as measured using Cu Kα1 radiation with an X-ray wavelength of 1.54060 Å.

In some embodiments, Form 2 of crystalline Compound A is characterized by a differential scanning calorimetry (DSC) thermogram substantially the same as shown in FIG. 5. In some embodiments, Form 2 of crystalline Compound A is characterized by a differential scanning calorimetry (DSC) thermogram comprising: an endotherm in the range of about 260-270 °C. In some embodiments. Form 2 of crystalline Compound A is characterized by a differential scanning calorimetry (DSC) thermogram comprising: an endotherm with an onset of about 265 °C and a peak of about 266 °C.

In some embodiments, Form 2 of crystalline Compound A is characterized by an endotherm in the range of about 260-270 °C for example at about 260-270 °C, 260-268 °C, 260-266 °C, 260-264 °C, 260-262 °C, 262-270 °C, 262-268 °C, 262-266 °C, 262-264 °C, 264-270 °C, 264-268 °C, 264-266 °C, 266-270 °C, 266-268 °C, or 268-270 °C in the DSC thermogram. In some embodiments, Form 2 of crystalline Compound A is characterized by an endotherm at about 266 °C in the DSC thermogram.

In some embodiments, Form 2 of crystalline Compound A is characterized by a Tihermogravimetric Analysis (TGA) thermogram substantially the same as shown in FIG. 6.

In some embodiments, Form 1 of crystalline Compound A is formed by exposing Form 2 of crystalline Compound A to ambient conditions. In some embodiments, Form 1 of crystalline Compound A is formed by exposing Form 2 of crystalline Compound A to humid conditions. In some embodiments, the humid condition comprises greater than about 25% RH. In some embodiments, the humid condition comprises greater than about 30% RH. In some embodiments, the humid condition comprises greater than about 35% RH. In some embodiments, the humid condition comprises greater than about 40% RH. In some embodiments, the humid condition comprises greater than about 45 RH. In some embodiments, the humid condition comprises greater than about 50% RH. In some embodiments, the humid condition comprises greater than about 55% RH. In some embodiments, the humid condition comprises greater than about 60% RH. In some embodiments, the humid condition comprises greater than about 65% RH. In some embodiments, the humid condition comprises greater than about 70% RH. In some embodiments, the humid condition comprises greater than about 75% RH. In some embodiments, the humid condition comprises greater than about 80% RH. In some embodiments, the humid condition comprises between about 30% and about 70% RH. In some embodiments, the humid condition comprises less than about 90% RH. In some embodiments, the humid condition comprises less than about 80% RH. In some embodiments, the humid condition comprises less than about 70% RH. In some embodiments, the humid condition comprises less than about 60% RH. In some embodiments, the humid condition comprises less than about 50% RH. In some embodiments, the humid condition comprises less than about 40% RH. In some embodiments, the humid condition comprises less than about 30% RH. In some embodiments, In some embodiments, Form 1 of crystalline Compound A is formed by a process comprising the steps of: (i) exposing Form 2 of crystalline Compound A to a first humidity; and (ii) exposing the compound from step (i) to a second humidity. In some embodiments, the first humidity is higher than the second humidity. In some embodiments, the first humidity is greater than about 80% RH. In some embodiments, the first humidity is greater than about 90% RH. In some embodiments, the first humidity is between about 80% and about 90% RH. In some embodiments, the second humidity is less than about 80% RH. In some embodiments, the second humidity is less than 70% RH. In some embodiments, the second humidity is between about 30% to about 70% RH. In some embodiments, the formation further comprises allowing crystalline Form 2 of Compound A to stand at a temperature between about 15 °C and about 80 °C. In some embodiments, the temperature is between about 20 °C and about 80 °C. In some embodiments, the temperature is between about 20 °C and about 75 °C. In some embodiments, the temperature is between about 25 °C and about 80 °C. In some embodiments, the temperature is between about 25 °C and about 75 °C. In some embodiments, the temperature is between about 25 °C and about 70 °C. In some embodiments, the temperature is between about 25 °C and about 65 °C. In some embodiments, the temperature is between about 20 °C and about 60 °C. In some embodiments, the temperature is greater than about 20 °C. In some embodiments, the temperature is greater than about 25 °C. In some embodiments, the temperature is greater than about 35 °C. In some embodiments, the temperature is greater than about 50 °C. In some embodiments, the temperature is greater than about 60 °C. In some embodiments, the temperature is greater than about 70 °C. In some embodiments, the temperature is less than about 80 °C. In some embodiments, the temperature is less than about 70 °C. In some embodiments, the temperature is less than about 65 °C. In some embodiments, the temperature is less than about 55 °C. In some embodiments, the temperature is less than about 45 °C. In some embodiments, the temperature is less than about 35 °C.

### Crystalline Compound A Form 3

FIG. 7 shows the X-ray powder diffraction (XRPD) pattern for crystalline Form 3 of crystalline Compound A.

In one aspect, provided herein is crystalline 5-(3,6-dibromo-9H-carbazol-9-yl)-N,N,N-trimethylpentan-1-aminium chloride (Compound A). Some embodiments provide a composition comprising crystalline 5-(3,6-dibromo-9H-carbazol-9-yl)-N,N,N-trimethylpentan-1-aminium chloride. In some embodiments, crystalline 5-(3,6-dibromo-9H-carbazol-9-yl)-N,N,N-trimethylpentan-1-aminium chloride Form 3 (Compound A Form 3) is characterized as having:
(a) an X-ray powder diffraction pattern comprising peaks at 28.4±0.2° 2-θ, 18.5±0.2° 2-θ, and 12.1±0.2° 2-θ, as measured using Cu Kα1 radiation with an X-ray wavelength of 1.54060 Å;
(b) an X-ray powder diffraction pattern substantially the same as shown in FIG. 7, as measured using Cu Kα1 radiation with an X-ray wavelength of 1.54060 Å;
or
(c) combinations thereof.

In some embodiments, Form 3 of crystalline Compound A is characterized as having an X-ray powder diffraction pattern substantially the same as shown in FIG. 7.

In some embodiments, Form 3 of crystalline Compound Ais characterized as having an X-ray powder diffraction pattern comprising peaks at 28.4±0.2° 2-θ, 18.5±0.2° 2-θ, and 12.1±0.2° 2-θ, as measured using Cu Kα1 radiation with an X-ray wavelength of 1.54060 Å. In some embodiments, Form 3 of crystalline Compound Ais characterized as having an X-ray powder diffraction pattern comprising peaks at 28.4±0.1° 2-θ, 18.5±0.1° 2-θ, and 12.1±0.1° 2-θ, as measured using Cu Kα1 radiation with an X-ray wavelength of 1.54060 Å. In some embodiments, Form 3 of crystalline Compound Ais characterized as having an X-ray powder diffraction pattern comprising peaks at about 28.4° 2-θ, about 18.5° 2-θ, and about 12.1° 2-θ, as measured using Cu Kα1 radiation with an X-ray wavelength of 1.54060 Å.

In some embodiments, the X-ray powder diffraction pattern of Form 3 of crystalline Compound A further comprises at least one peak selected from 26.8±0.2° 2-θ, 31.7±0.2° 2-θ, and 23.3±0.2° 2-θ, as measured using Cu Kα1 radiation with an X-ray wavelength of 1.54060 Å. In some embodiments, the X-ray powder diffraction pattern of Form 3 of crystalline Compound A further comprises at least one peak selected from 26.8±0.1° 2-θ, 31.7±0.1° 2-θ, and 23.3±0.1° 2-θ, as measured using Cu Kα1 radiation with an X-ray wavelength of 1.54060 Å. In some embodiments, the X-ray powder diffraction pattern of Form 3 of crystalline Compound A further comprises at least one peak selected from about 26.8° 2-θ, about 31.7° 2-θ, and about 23.3° 2-θ, as measured using Cu Kα1 radiation with an X-ray wavelength of 1.54060 Å.

In some embodiments, the X-ray powder diffraction pattern of Form 3 of crystalline Compound A further comprises at least one peak selected from 20.7±0.2° 2-θ, 30.8±0.2° 2-θ, and 21.8±0.2° 2-θ, as measured using Cu Kα1 radiation with an X-ray wavelength of 1.54060 Å. In some embodiments, the X-ray powder diffraction pattern of Form 3 of crystalline Compound A further comprises at least one peak selected from 20.7±0.1° 2-θ, 30.8±0.1° 2-θ, and 21.8±0.1° 2-θ, as measured using Cu Kα1 radiation with an X-ray wavelength of 1.54060 Å. In some embodiments, the X-ray powder diffraction pattern of Form 3 of crystalline Compound A further comprises at least one peak selected from about 20.7° 2-θ, about 30.8° 2-θ, and about 21.8° 2-θ, as measured using Cu Kα1 radiation with an X-ray wavelength of 1.54060 Å.

In some embodiments, the X-ray powder diffraction pattern comprises of Form 3 of crystalline Compound A at least one peak selected from 12.1±0.2° 2-θ, 18.5±0.2° 2-θ, 20.7±0.2° 2-θ, 21.8±0.2° 2-θ, 23.3±0.2° 2-θ, 26.8±0.2° 2-θ, 28.4±0.2° 2-θ, 30.8±0.2° 2-θ, and 31.7±0.2° 2-θ, as measured using Cu Kα1 radiation with an X-ray wavelength of 1.54060 Å. In some embodiments, the X-ray powder diffraction pattern of Form 3 of crystalline Compound A comprises at least two peaks selected from 12.1±0.2° 2-θ, 18.5±0.2° 2-θ, 20.7±0.2° 2-θ, 21.8±0.2° 2-θ, 23.3±0.2° 2-θ, 26.8±0.2° 2-θ, 28.4±0.2° 2-θ, 30.8±0.2° 2-θ, and 31.7±0.2° 2-θ, as measured using Cu Kα1 radiation with an X-ray wavelength of 1.54060 Å. In some embodiments, the X-ray powder diffraction pattern of Form 3 of crystalline Compound A comprises at least three peaks selected from 12.1±0.2° 2-θ, 18.5±0.2° 2-θ, 20.7±0.2° 2-θ, 21.8±0.2° 2-θ, 23.3±0.2° 2-θ, 26.8±0.2° 2-θ, 28.4±0.2° 2-θ, 30.8±0.2° 2-θ, and 31.7±0.2° 2-θ, as measured using Cu Kα1 radiation with an X-ray wavelength of 1.54060 Å. In some embodiments, the X-ray powder diffraction pattern of Form 3 of crystalline Compound A comprises at least four peaks selected from 12.1±0.2° 2-θ, 18.5±0.2° 2-θ, 20.7±0.2° 2-θ, 21.8±0.2° 2-θ, 23.3±0.2° 2-θ, 26.8±0.2° 2-θ, 28.4±0.2° 2-θ, 30.8±0.2° 2-θ, and 31.7±0.2° 2-θ, as measured using Cu Kα1 radiation with an X-ray wavelength of 1.54060 Å. In some embodiments, the X-ray powder diffraction pattern of Form 3 of crystalline Compound A comprises at least five peaks selected from 12.1±0.2° 2-θ, 18.5±0.2° 2-θ, 20.7±0.2° 2-θ, 21.8±0.2° 2-θ, 23.3±0.2° 2-θ, 26.8±0.2° 2-θ, 28.4±0.2° 2-θ, 30.8±0.2° 2-θ, and 31.7±0.2° 2-θ, as measured using Cu Kα1 radiation with an X-ray wavelength of 1.54060 Å. In some embodiments, the X-ray powder diffraction pattern of Form 3 of crystalline Compound A comprises at least six peaks selected from 12.1±0.2° 2-θ, 18.5±0.2° 2-θ, 20.7±0.2° 2-θ, 21.8±0.2° 2-θ, 23.3±0.2° 2-θ, 26.8±0.2° 2-θ, 28.4±0.2° 2-θ, 30.8±0.2° 2-θ, and 31.7±0.2° 2-θ, as measured using Cu Kα1 radiation with an X-ray wavelength of 1.54060 Å. In some embodiments, the X-ray powder diffraction pattern of Form 3 of crystalline Compound A comprises at least seven peaks selected from 12.1±0.2° 2-θ, 18.5±0.2° 2-θ, 20.7±0.2° 2-θ, 21.8±0.2° 2-θ, 23.3±0.2° 2-θ, 26.8±0.2° 2-θ, 28.4±0.2° 2-θ, 30.8±0.2° 2-θ, and 31.7±0.2° 2-θ, as measured using Cu Kα1 radiation with an X-ray wavelength of 1.54060 Å. In some embodiments, the X-ray powder diffraction pattern of Form 3 of crystalline Compound A comprises at least eight peaks selected from 12.1±0.2° 2-θ, 18.5±0.2° 2-θ, 20.7±0.2° 2-θ, 21.8±0.2° 2-θ, 23.3±0.2° 2-θ, 26.8±0.2° 2-θ, 28.4±0.2° 2-θ, 30.8±0.2° 2-θ, and 31.7±0.2° 2-θ, as measured using Cu Kα1 radiation with an X-ray wavelength of 1.54060 Å. In some embodiments, the X-ray powder diffraction pattern of Form 3 of crystalline Compound A comprises peaks at 12.1±0.2° 2-θ, 18.5±0.2° 2-θ, 20.7±0.2° 2-θ. 21.8±0.2° 2-θ, 23.3±0.2° 2-θ, 26.8±0.2° 2-θ, 28.4±0.2° 2-θ, 30.8±0.2° 2-θ, and 31.7±0.2° 2-θ, as measured using Cu Kα1 radiation with an X-ray wavelength of 1.54060 Å. In some embodiments, the X-ray powder diffraction pattern of Form 3 of crystalline Compound A comprises peaks at 12.1±0.1° 2-θ, 18.5±0.1° 2-θ, 20.7±0.1° 2-θ, 21.8±0.1° 2-θ, 23.3±0.1° 2-θ, 26.8±0.1° 2-θ, 28.4±0.1° 2-θ, 30.8±0.1° 2-θ, and 31.7±0.1° 2-θ, as measured using Cu Kα1 radiation with an X-ray wavelength of 1.54060 Å. In some embodiments, the X-ray powder diffraction pattern of Form 3 of crystalline Compound A comprises peaks at about 12.1° 2-θ, about 18.5° 2-θ, about 20.7° 2-θ, about 21.8° 2-θ, about 23.3° 2-θ, about 26.8° 2-θ, about 28.4° 2-θ, about 30.8° 2-θ, and about 31.7° 2-θ, as measured using Cu Kα1 radiation with an X-ray wavelength of 1.54060 Å.

### Crystalline Compound A Form 4

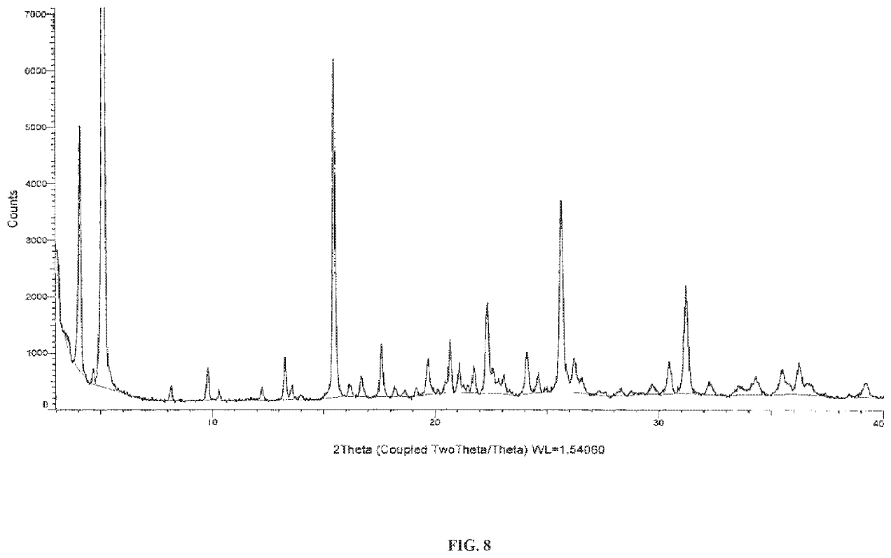

FIG. 8 shows the X-ray powder diffraction (XRPD) pattern for Form 4 of crystalline Compound A.

In one aspect, provided herein is crystalline 5-(3,6-dibromo-9H-carbazol-9-yl)-N,N,N-trimethylpentan-1-aminium chloride (Compound A). Some embodiments provide a composition comprising crystalline 5-(3,6-dibromo-9H-carbazol-9-yl)-N,N,N-trimethylpentan-1-aminium chloride. In some embodiments, crystalline 5-(3,6-dibromo-9H-carbazol-9-yl)-N,N,N-trimethylpentan-1-aminium chloride Form 4 (Compound A Form 4) is characterized as having:
(a) an X-ray powder diffraction pattern comprising peaks at 3.0±0.2° 2-θ, 22.3±0.2° 2-θ, and 31.2±0.2° 2-θ, as measured using Cu Kα1 radiation with an X-ray wavelength of 1.54060 Å;
(b) an X-ray powder diffraction pattern substantially the same as shown in FIG. 8;
or
(c) combinations thereof.

In some embodiments, Form 4 of crystalline Compound A is characterized as having an X-ray powder diffraction pattern substantially the same as shown in FIG. 8.

In some embodiments, Form 4 of crystalline Compound A is characterized as having an X-ray powder diffraction pattern comprising peaks at 3.0±0.2° 2-θ, 22.3±0.2° 2-θ, and 31.2±0.2° 2-θ, as measured using Cu Kα1 radiation with an X-ray wavelength of 1.54060 Å. In some embodiments, Form 4 of crystalline Compound A is characterized as having an X-ray powder diffraction pattern comprising peaks at 3.0±0.1° 2-θ, 22.3±0.1° 2-θ, and 31.2±0.1° 2-θ, as measured using Cu Kai radiation with an X-ray wavelength of 1.54060 Å. In some embodiments, Form 4 of crystalline Compound A is characterized as having an X-ray powder diffraction pattern comprising peaks at about 3.0° 2-θ, about 22.3° 2-θ, and about 31.2° 2-θ, as measured using Cu Kα1 radiation with an X-ray wavelength of 1.54060 Å.

In some embodiments, Form 4 of crystalline Compound A is characterized as having an X-ray powder diffraction pattern comprising peaks at 4.0±0.2° 2-θ, 5.0±0.2° 2-θ, and 15.3±0.2° 2-θ, as measured using Cu Kα1 radiation with an X-ray wavelength of 1.54060 Å. In some embodiments, Form 4 of crystalline Compound A is characterized as having an X-ray powder diffraction pattern comprising peaks at 4.0±0.1° 2-θ, 5.0±0.10 2-θ, and 15.3±0.1° 2-θ, as measured using Cu Kα1 radiation with an X-ray wavelength of 1.54060 Å. In some embodiments, Form 4 of crystalline Compound A is characterized as having an X-ray powder diffraction pattern comprising peaks at about 4.0° 2-θ, about 5.0° 2-θ, and about 15.3° 2-θ, as measured using Cu Kα1 radiation with an X-ray wavelength of 1.54060 Å.

In some embodiments, the X-ray powder diffraction pattern of Form 4 of crystalline Compound A further comprises at least one peak selected from 3.0±0.2° 2-θ, 25.6±0.2° 2-θ, and 31.2f 0.2° 2-θ, as measured using Cu Kα1 radiation with an X-ray wavelength of 1.54060 Å. In some embodiments, the X-ray powder diffraction pattern of Form 4 of crystalline Compound A further comprises at least one peak selected from 3.0±0.1° 2-θ, 25.6±0.1° 2-θ, and 31.2±0.1° 2-θ, as measured using Cu Kα1 radiation with an X-ray wavelength of 1.54060 Å. In some embodiments, the X-ray powder diffraction pattern of Form 4 of crystalline Compound A further comprises at least one peak selected from about 3.0° 2-θ, about 25.6° 2-θ, and about 31.2° 2-θ, as measured using Cu Kα1 radiation with an X-ray wavelength of 1.54060 Å.

In some embodiments, the X-ray powder diffraction pattern of Form 4 of crystalline Compound A further comprises at least one peak selected from 17.6±0.2° 2-θ, 20.6±0.2° 2-θ, and 22.3±0.2° 2-θ, as measured using Cu Kα1 radiation with an X-ray wavelength of 1.54060 Å. In some embodiments, the X-ray powder diffraction pattern of Form 4 of crystalline Compound A further comprises at least one peak selected from 17.6±0.1° 2-θ, 20.6±0.1° 2-θ, and 22.3±0.1° 2-θ, as measured using Cu Kα1 radiation with an X-ray wavelength of 1.54060 Å. In some embodiments, the X-ray powder diffraction pattern of Form 4 of crystalline Compound A further comprises at least one peak selected from about 17.6° 2-θ, about 20.6° 2-θ, and about 22.3° 2-θ, as measured using Cu Kα1 radiation with an X-ray wavelength of 1.54060 Å.

In some embodiments, the X-ray powder diffraction pattern of Form 4 of crystalline Compound A comprises at least one peak selected from 3.0±0.2° 2-θ, 4.0±0.2° 2-θ, 5.0±0.2° 2-θ, 15.3±0.2° 2-θ, 17.6±0.2° 2-θ, 20.6±0.2° 2-θ, 22.3±0.2° 2-θ, 25.6±0.2° 2-θ, and 31.2±0.2° 2-θ, as measured using Cu Kα1 radiation with an X-ray wavelength of 1.54060 Å. In some embodiments, the X-ray powder diffraction pattern of Form 4 of crystalline Compound A comprises at least two peaks selected from 3.0±0.2° 2-θ. 4.0±0.2° 2-θ, 5.0±0.2° 2-θ, 15.3±0.2° 2-θ, 17.6±0.2° 2-θ, 20.6±0.2° 2-θ, 22.3±0.2° 2-θ, 25.6±0.2° 2-θ, and 31.2±0.2° 2-θ, as measured using Cu Kα1 radiation with an X-ray wavelength of 1.54060 Å. In some embodiments, the X-ray powder diffraction pattern of Form 4 of crystalline Compound A comprises at least three peaks selected from 3.0±0.2° 2-θ, 4.0±0.2° 2-θ, 5.0±0.2° 2-θ, 15.3±0.2° 2-θ, 17.6±0.2° 2-θ, 20.6±0.2° 2-θ, 22.3±0.2° 2-θ, 25.6±0.2° 2-θ, and 31.2±0.2° 2-θ, as measured using Cu Kα1 radiation with an X-ray wavelength of 1.54060 Å. In some embodiments, the X-ray powder diffraction pattern of Form 4 of crystalline Compound A comprises at least four peaks selected from 3.0±0.2° 2-θ, 4.0±0.2° 2-θ, 5.0±0.2° 2-θ, 15.3±0.2° 2-θ, 17.6±0.2° 2-θ, 20.6±0.2° 2-θ, 22.3±0.2° 2-θ, 25.6±0.2° 2-θ, and 31.2±0.2° 2-θ, as measured using Cu Kα1 radiation with an X-ray wavelength of 1.54060 Å. In some embodiments, the X-ray powder diffraction pattern of Form 4 of crystalline Compound A comprises at least five peaks selected from 3.0±0.2° 2-θ, 4.0±0.2° 2-θ, 5.0±0.2° 2-θ, 15.3±0.2° 2-θ, 17.6±0.2° 2-θ, 20.6±0.2° 2-θ, 22.3±0.2° 2-θ, 25.6±0.2° 2-θ, and 31.2±0.2° 2-θ, as measured using Cu Kα1 radiation with an X-ray wavelength of 1.54060 Å. In some embodiments, the X-ray powder diffraction pattern of Form 4 of crystalline Compound A comprises at least six peaks selected from 3.0±0.2° 2-θ, 4.0±0.2° 2-θ, 5.0±0.2° 2-θ, 15.3±0.2° 2-θ, 17.6±0.2° 2-θ, 20.6±0.2° 2-θ, 22.3±0.2° 2-θ, 25.6±0.2° 2-θ, and 31.2±0.2° 2-θ, as measured using Cu Kα1 radiation with an X-ray wavelength of 1.54060 Å. In some embodiments, the X-ray powder diffraction pattern of Form 4 of crystalline Compound A comprises at least seven peaks selected from 3.0±0.2° 2-θ, 4.0±0.2° 2-θ, 5.0±0.2° 2-θ, 15.3±0.2° 2-θ, 17.6±0.2° 2-θ, 20.6±0.2° 2-θ, 22.3±0.2° 2-θ, 25.6±0.2° 2-θ, and 31.2±0.2° 2-θ, as measured using Cu Kα1 radiation with an X-ray wavelength of 1.54060 Å. In some embodiments, the X-ray powder diffraction pattern of Form 4 of crystalline Compound A comprises at least eight peaks selected from 3.0±0.2° 2-θ, 4.0±0.2° 2-θ, 5.0±0.2° 2-θ, 15.3±0.2° 2-θ, 17.6±0.2° 2-θ, 20.6±0.2° 2-θ, 22.3±0.2° 2-θ, 25.6±0.2° 2-θ, and 31.2±0.2° 2-θ, as measured using Cu Kα1 radiation with an X-ray wavelength of 1.54060 Å. In some embodiments, the X-ray powder diffraction pattern of Form 4 of crystalline Compound A comprises peaks at 3.0±0.2° 2-θ, 4.0±0.2° 2-θ, 5.0±0.2° 2-θ, 15.3±0.2° 2-θ, 17.6±0.2° 2-θ, 20.6±0.2° 2-θ, 22.3±0.2° 2-θ, 25.6±0.2° 2-θ, and 31.2±0.2° 2-θ, as measured using Cu Kα1 radiation with an X-ray wavelength of 1.54060 Å. In some embodiments, the X-ray powder diffraction pattern of Form 4 of crystalline Compound A comprises peaks at 3.0±0.1° 2-θ, 4.0±0.1° 2-θ, 5.0±0.1° 2-θ, 15.3±0.1° 2-θ, 17.6±0.1° 2-θ, 20.6±0.1° 2-θ, 22.3±0.1° 2-θ, 25.6±0.1° 2-θ, and 31.2±0.1° 2-θ, as measured using Cu Kα1 radiation with an X-ray wavelength of 1.54060 Å. In some embodiments, the X-ray powder diffraction pattern of Form 4 of crystalline Compound A comprises peaks at about 3.0° 2-θ, about 4.0° 2-θ, about 5.0° 2-θ, about 15.3° 2-θ, about 17.6° 2-θ, about 20.6° 2-θ, about 22.3° 2-θ, about 25.6° 2-θ, and about 31.2° 2-θ, as measured using Cu Kα1 radiation with an X-ray wavelength of 1.54060 Å.

In some embodiments, Form 1 of crystalline Compound A is formed by exposing Form 4 of crystalline Compound A to humid conditions. In some embodiments, the humid condition comprises greater than about 25% RH. In some embodiments, the humid condition comprises greater than about 30% RH. In some embodiments, the humid condition comprises greater than about 35% RH. In some embodiments, the humid condition comprises greater than about 40% RH. In some embodiments, the humid condition comprises greater than about 45% RH. In some embodiments, the humid condition comprises greater than about 50% RH. In some embodiments, the humid condition comprises greater than about 55% RH. In some embodiments, the humid condition comprises greater than about 60% RH. In some embodiments, the humid condition comprises greater than about 65% RH. In some embodiments, the humid condition comprises greater than about 70% RH. In some embodiments, the humid condition comprises greater than about 75% RH. In some embodiments, the humid condition comprises greater than about 80% RH. In some embodiments, the humid condition comprises between about 30% and about 70% RH. In some embodiments, the humid condition comprises less than about 90% RH. In some embodiments, the humid condition comprises less than about 80% RH. In some embodiments, the humid condition comprises less than about 70% RH. In some embodiments, the humid condition comprises less than about 60% RH. In some embodiments, the humid condition comprises less than about 50% RH. In some embodiments, the humid condition comprises less than about 40% RH. In some embodiments, the humid condition comprises less than about 30% RH. In some embodiments, the formation further comprises allowing crystalline Form 4 of Compound A to stand at a temperature between about 15 °C and about 80 °C. In some embodiments, the temperature is between about 20 °C and about 80 °C. In some embodiments, the temperature is between about 20 °C and about 75 °C. In some embodiments, the temperature is between about 25 °C and about 80 °C. In some embodiments, the temperature is between about 25 °C and about 75 °C. In some embodiments, the temperature is between about 25 °C and about 70 °C. In some embodiments, the temperature is between about 25 °C and about 65 °C. In some embodiments, the temperature is between about 20 °C and about 60 °C. In some embodiments, the temperature is between about 25 °C and about 40 °C. In some embodiments, the temperature is greater than about 20 °C. In some embodiments, the temperature is greater than about 25 °C. In some embodiments, the temperature is greater than about 35 °C. In some embodiments, the temperature is greater than about 50 °C. In some embodiments, the temperature is greater than about 60 °C. In some embodiments, the temperature is greater than about 70 °C. In some embodiments, the temperature is less than about 80 °C. In some embodiments, the temperature is less than about 70 °C. In some embodiments, the temperature is less than about 65 °C. In some embodiments, the temperature is less than about 55 °C. In some embodiments, the temperature is less than about 45 °C. In some embodiments, the temperature is less than about 35 °C.

### II. METHODS OF MAKING COMPOUND A AND POLYMORPHIC FORM THEREOF

In one aspect, the invention provides methods of making a crystalline form of Compound A:

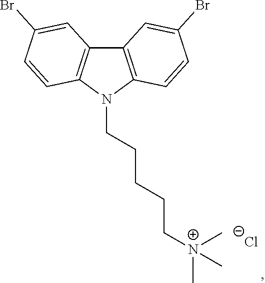 [SMILES: C[N+](C)(C)CCCCCn1c2ccc(Br)cc2c2cc(Br)ccc21 ; [Cl-]]

In some embodiments, Compound A is prepared according to the examples herein.

The crystalline Compound A according to the invention is not limited by the starting materials used to produce Compound A.

In one aspect, the invention is directed to methods of making a crystalline form of Compound A, or a pharmaceutically acceptable salt and/or solvate thereof, either by isolation of the desired crystalline form as the first solid form after synthesis of Compound A, or alternatively, by isolation of the desired crystalline form as a transition from a prior solid form of Compound A. Transitions from one form to another are within the scope of the invention because they can be an alternative manufacturing method for obtaining the form desired for the production of the medicinal preparations.

Isolation and purification of the chemical entities and intermediates described herein can be performed, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be found by referencing the examples below. However, other equivalent separation or isolation procedures can also be used. Prior to crystallization, Compound A may be isolated in about 50% chemical purity, 55% chemical purity, 60% chemical purity, 65% chemical purity, 70% chemical purity, 75% chemical purity, 80% chemical purity, 90% chemical purity, 91% chemical purity, 92% purity, 93% chemical purity, 94% chemical purity, 95% chemical purity, 96% chemical purity, 97% chemical purity, 98% chemical purity, 99% chemical purity, about 98% chemical purity, or about 100% chemical purity.

In some embodiments, the crystalline form disclosed herein is obtained by crystallizing Compound A with a chemical purity of less than about 98%, less than about 97%, less than about 96%, less than about 95%, less than about 94%, less than about 93%, less than about 92%, less than about 91%, less than about 90%, less than about 89%, less than about 88%, less than about 87%, less than about 86%, less than about 85%, less than about 84%, less than about 83%, less than about 82%, less than about 81%, less than about 80%, less than about 78%, less than about 76%, less than about 74%, less than about 72%, or less than about 70%. In some embodiments, the crystalline form is obtained by crystallizing Compound A with a chemical purity in the range of about 70% to about 99%, 80% to about 96%, about 85% to about 96%, about 90% to about 96%, about 80% to 98%, about 85% to about 98%, about 90% to about 98%, about 92% to about 98%, about 94% to 98%, or about 96% to about 98%.

In some embodiments, isolating the desired crystalline form of Compound A involves crystallization of crude reaction product from a mono-solvent system. In various embodiments, isolating the desired crystalline form of Compound A involves crystallization of crude product from a binary, tertiary, or greater solvent system, collectively understood as a multi-solvent system.

In some embodiments, the crystallization is carried out by generating the desired Compound A in a reaction mixture and isolating the desired crystalline form from the reaction mixture. In some embodiments, the reaction mixture is formed by adding (5-bromopentyl)-trimethyl-ammonium bromide, potassium carbonate, to a solution of 3,6-dibromocarbazole to form dissolved 5-(3,6-dibromo-9H-carbazol-9-yl)-N,N,N-trimethylpentan-1-aminium chloride (Compound A). In other embodiments, the reaction mixture is formed by dissolving Compound A into a solvent. In some embodiments, brine and HCl are also added to the reaction mixture. In some embodiments, the brine and HCl are added to the reaction mixture after reaction completion.

### Preparation of Crystalline Compound A

In some embodiments, crystalline Compound A is obtained from a solution formed by addition of a solvent to Compound A. In some embodiments, the solvent is selected from 1-butanol, 2-propanol, acetone, acetonitrile, benzyl alcohol, dichloromethane, dimethylsulfoxide, ethanol, ethyl acetate, isopropyl acetate, methanol, methyl ethyl ketone, propylene glycol, methyl isobutyl ketone, tert-butyl methyl ether, tetrahydrofuran, toluene. Tween 80, water, and combinations thereof.

In some embodiments, crystalline Compound A is obtained by temperature cycling a solution of Compound A. In some embodiments, the temperature cycling comprises cycling the temperature between 0 °C and 80 °C, such as between 0 °C and 80 °C, 0 °C and 70 °C, 0 °C and 60 °C, 0 °C and 50 °C, 5 °C and 80 °C, 5 °C and 70 °C, 5 °C and 60 °C, 5 °C and 50 °C, 10 °C and 80 °C, 10 °C and 70 °C, 10 °C and 60 °C, 10 °C and 50 °C, 15 °C and 80 °C, 15 °C and 70 °C, 15 °C and 60 °C, 15 °C and 50 °C, 20 °C and 80 °C, 20 °C and 70 °C, 20 °C and 60 °C, or 20 °C and 50 °C. In some embodiments, the temperature cycling comprises cycling the temperature between 5 °C and 50 °C. In some embodiments, the temperature cycling comprises between 0.5 and 10 hour hold periods at the low and elevated temperatures, such as between 0.5 and 10 hours, 0.5 and 8 hours, 0.5 and 6 hours, 0.5 and 4 hours, 0.5 and 2 hours, 1 and 10 hours, 1 and 8 hours, 1 and 6 hours, 1 and 4 hours, 1 and 2 hours, 2 and 10 hours, 2 and 8 hours, 2 and 6 hours, 2 and 4 hours, 4 and 10 hours, 4 and 8 hours, 4 and 6 hours, 6 and 10 hours, 6 and 8 hours, or 8 and 10 hours. In some embodiments, the temperature cycling comprises 1 hour hold periods at the low and elevated temperatures. In some embodiments, the temperature cycling further comprises addition of an anti-solvent. In some embodiments, the anti-solvent is tert-butyl methyl ether.

In some embodiments, crystalline Compound A is obtained by crash cooling a solution of Compound A. In some embodiments, the crash cooling comprises rapidly cooling the solution to a temperature of between 0 °C and 10 °C, such as between 0 °C and 10 °C, 0 °C and 8 °C, 0 °C and 6 °C, 0 °C and 4 °C, 0 °C and 2 °C, 1 °C and 10 °C, 1 °C and 8 °C, 1 °C and 6 °C, 1 °C and 4 °C, 1 °C and 2 °C, 2 °C and 10 °C, 2 °C and 8 °C, 2 °C and 6 °C, 2 °C and 4 °C, 4 °C and 10 °C, 4 °C and 8 C, 4 °C and 6 °C, 6 °C and 10 °C, 6 °C and 8 °C or 8 °C and 10 °C. In some embodiments, the crash cooling comprises rapidly cooling the solution to a temperature of 6 °C. In some embodiments, the crash cooling further comprises rapidly cooling the solution to a temperature of between 0 °C and −20 °C, such as between 0 °C and −20 °C, 0 °C and −18 °C, 0 °C and −16 °C, 0 °C and −14 °C, 0 °C and −12 °C, 0 °C and −10 °C, 0 °C and −8 °C, 0 °C and −6 °C, 0 °C and −4 °C, 0 °C and −2 °C, −2 °C and −20 °C, −2 °C and −18 °C, −2 °C and −16 °C, −2 °C and −14 °C, −2 °C and −12 °C, −2 °C and −10 °C, −2 °C and −8 °C, −2 °C and −6 °C, −2 °C and −4 °C, −4 °C and −20 °C, −4 °C and −18 °C, −4 °C and −16 °C, −4 °C and −14 °C, −4 °C and −12 °C, −4 °C and −10 °C, −4 °C and −8 °C, −4 °C and −6 °C, −6 °C and −20 °C, −6 °C and −18 °C, −6 °C and −16 °C, −6 °C and −14 °C, −6 °C and −12 °C, −6 °C and −10 °C, −6 °C and −8 °C, −8 °C and −20 °C, −8 °C and −18 °C, −8 °C and −16 °C, −8 °C and −14 °C, −8 °C and −12 °C, −8 °C and −10 °C, −10 °C and −20 °C, −10 °C and −18 °C, −10 °C and −16 °C, −10 °C and −14 °C, −10 °C and −12 °C, −12 °C and −20 °C, −12 °C and −18 °C, −12 °C and −16 °C, −12 °C and −14 °C, −14 °C and −20 °C, −14 °C and −18 °C, −14 °C and −16 °C, −16 °C and −20 °C, −16 °C and −18 °C, or −18 °C and −20 °C. In some embodiments, the crash cooling further comprises rapidly cooling the solution to a temperature of −18 °C. In some embodiments, the crash cooling further comprises addition of an anti-solvent. In some embodiments, the anti-solvent is added prior to cooling. In some embodiments, the anti-solvent is added after cooling. In some embodiments, the anti-solvent is added after a first cooling and before a second cooling. In some embodiments, the anti-solvent is tert-butyl methyl ether.

In various embodiments, the crystallization further involves filtering the solution containing the obtained crystals of crystalline Compound A. In some embodiments, the crystallization optionally involves washing the obtained crystals by a solvent, for example by the recrystallization solvent one or more times. In some embodiments, the crystallization optionally involves drying the obtained crystals, for example under vacuum at a temperature of about 20-30 °C.

In some embodiments, the chemical purity of crystalline Compound A is greater than 60%, 70%, 80%, 90%, 95%, or 99%. In some embodiments, the chemical purity of crystalline Compound A is greater than about 90%. In some embodiments, the chemical purity of crystalline Compound A is greater than about 95%. In some embodiments, the chemical purity of crystalline Compound A greater than about 99%. The chemical purity of crystalline Compound A may be measured by any available analytical technique, for example by HPLC analysis.

In various embodiments, crystalline Compound A is solvated. In some embodiments, crystalline Compound A is anhydrous (i.e. not hydrated). In various embodiments, crystalline Compound A is hydrated. In some embodiments, crystalline Compound A is hydrated and characterized as having 1 to 2 water molecules associated with the hydrate. In some embodiments, crystalline Compound A is hydrated, and the average number of water molecules associated with the hydrate is about 1.5. In some embodiments, Compound A is multiply hydrated. In various embodiments, crystalline Compound A is a dihydrate. In some embodiments, crystalline Compound A comprises water content. In some embodiments, crystalline Compound A comprises about 1 to about 10% water content (weight/weight %). In some embodiments, crystalline Compound A comprises about 1%, about 2%, about 3%, about 4%, about 5%, about 6%, about 7%, about 8%, about 9%, or about 10% (w/w %) water content. In some embodiments, crystalline Compound A comprises 2 to about 8% water content (w/w %). In some embodiments, crystalline Compound A comprises 3 to about 6% water content (w/w %). In some embodiments, crystalline Compound A comprises 3.5 to about 5.5% water content (w/w %). In some embodiments, the water content is determined by Karl Fischer coulometric titration.

### III. ADDITIONAL DEFINITIONS

As used herein, “active agent” is used to indicate a chemical entity which has biological activity. In certain embodiments, an “active agent” is a compound having pharmaceutical utility.

As used herein, “modulation” refers to a change in activity as a direct or indirect response to the presence of a chemical entity as described herein, relative to the activity of in the absence of the chemical entity. The change may be an increase in activity or a decrease in activity, and may be due to the direct interaction of the compound with the target or due to the interaction of the compound with one or more other factors that in turn affect the target's activity. For example, the presence of the chemical entity may, for example, increase or decrease the target activity by directly binding to the target, by causing (directly or indirectly) another factor to increase or decrease the target activity, or by (directly or indirectly) increasing or decreasing the amount of target present in the cell or organism.

As used herein, “therapeutically effective amount” of a chemical entity described herein refers to an amount, when administered to a human or non-human subject, that provides a therapeutic benefit such as amelioration of symptoms, slowing of disease progression, or prevention of disease.

“Treating” or “treatment” encompasses administration of Compound A, or a pharmaceutically acceptable salt, solvate, or hydrate thereof, to a mammalian subject, particularly a human subject, in need of such an administration and includes (i) arresting the development of clinical symptoms of the disease, (ii) bringing about a regression in the clinical symptoms of the disease, and/or (iii) prophylactic treatment for preventing the onset of the disease.

As used herein, a “pharmaceutically acceptable” component is one that is suitable for use with humans and/or animals without undue adverse side effects (such as toxicity, irritation, and allergic response) commensurate with a reasonable benefit/risk ratio.

“Pharmaceutically acceptable salts” include, but are not limited to salts with inorganic acids, such as hydrochloride, carbonate, phosphate, hydrogenphosphate, diphosphate, hydrobromide, sulfate, sulfinate, nitrate, and like salts; as well as salts with an organic acid, such as malate, malonate, maleate, fumarate, tartrate, succinate, citrate, acetate, lactate, gluconate, methanesulfonate, Tris (hydroxymethyl-aminomethane), p-toluenesulfonate, priopionate, 2-hydroxyethylsulfonate, benzoate, salicylate, stearate, oxalate, pamoate, and alkanoate such as acetate, HO₂C—(CH₂)ₙ—CO₂H where n is 0-4, and like salts. Other salts include sulfate, methasulfonate, chloride, bromide, trifluoracetate, picrate, sorbate, benzilate, salicilate, nitrate, phthalate or morpholine.

As used herein, “subject” refers to a mammal that has been or will be the object of treatment, observation or experiment. The methods described herein can be useful in both human therapy and veterinary applications. In some embodiments, the subject is a human.

The term “mammal” is intended to have its standard meaning, and encompasses humans, dogs, cats, sheep, and cows, for example.

The compounds disclosed herein can be used in different enriched isotopic forms, e.g., enriched in the content of ²H, ³H, ¹¹C, ¹³C and/or ¹⁴C. In one particular embodiment, the compound is deuterated at least one position. Such deuterated forms can be made by the procedure described in U.S. Pat. Nos. 5,846,514 and 6,334,997. As described in U.S. Pat. Nos. 5,846,514 and 6,334,997, deuteration can improve the efficacy and increase the duration of action of drugs.

Deuterium substituted compounds can be synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varna, Rajender S. The Synthesis of Radiolabeled Compounds via Organometallic Intermediates, Tetrahedron. 1989, 45(21), 6601-21; and Evans, E. Anthony. Synthesis of radiolabeled compounds. J. Radioanal. Chem., 1981, 64(1-2), 9-32.

In one aspect, described is a compound with the following structure:

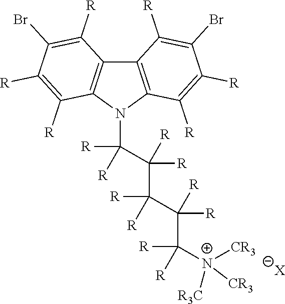

wherein,
each R is independently selected from hydrogen or deuterium, and
X is an anion.
   In some embodiments, X is a halide (Cl, Br, or I). In some embodiments, X is Cl.

A “solvate” is formed by the interaction of a solvent and a compound. The term “compound” is intended to include solvates of compounds. Similarly, “pharmaceutically acceptable salts” includes solvates of pharmaceutically acceptable salts. Suitable solvates are pharmaceutically acceptable solvates, such as hydrates, including monohydrates and hemi-hydrates. Also included are solvates formed with the one or more crystallization solvents.

Pharmaceutically acceptable forms of the compounds recited herein include pharmaceutically acceptable salts, chelates, non-covalent complexes, prodrugs, and mixtures thereof.

A “chelate” is formed by the coordination of a compound to a metal ion at two (or more) points. The term “compound” is intended to include chelates of compounds. Similarly, “pharmaceutically acceptable salts” includes chelates of pharmaceutically acceptable salts.

A “non-covalent complex” is formed by the interaction of a compound and another molecule wherein a covalent bond is not formed between the compound and the molecule. For example, complexation can occur through van der Waals interactions, hydrogen bonding, and electrostatic interactions (also called ionic bonding). Such non-covalent complexes are included in the term “compound”. Similarly, pharmaceutically acceptable salts include “non-covalent complexes” of pharmaceutically acceptable salts.

When ranges are used herein for physical properties, such as molecular weight, or chemical properties, such as chemical formulae, all combinations and sub combinations of ranges and specific embodiments therein are intended to be included.

The term “about” when referring to a number or a numerical range means that the number or numerical range referred to is an approximation within experimental variability (or within statistical experimental error), and thus the number or numerical range may vary from, for example, between 1% and 15% of the stated number or numerical range. In some instances of numerical ranges, “about” means ±10%.

As used herein, “significant” refers to any detectable change that is statistically significant in a standard parametric test of statistical significance such as Student's T-test, where p<0.05.

### IV. METHODS OF USE

In another aspect, provided herein is a method of treating a disease or disorder in a subject in need thereof, comprising administering to the subject a therapeutically effective amount of the crystalline 5-(3,6-dibromo-9H-carbazol-9-yl)-N,N,N-trimethylpentan-1-aminium chloride, or hydrate thereof, provided herein or the pharmaceutical composition provided herein.

In some embodiments, the disease or disorder is a fat-related disorder. In some embodiments, the fat-related disorder is obesity, abnormal fat distribution, diabetes, a cardiovascular disease, obstructive sleep apnea, lipoma, cancer, osteoarthritis, an endocrinologic disease, a reproductive disease, a neurological disease, a psychiatric disease, a rheumatological disease, or an orthopedic disease. In some embodiments, the fat-related disorder is cellulite, excess fat in various body areas, lipoma, fatty tumor disease, a disorder associated with fat accumulation, Dercum's disease, lipedema, or hibernoma. In some embodiments, the fat-related disorder is cellulite. In some embodiments, the fat-related disorder is excess fat in various body areas. In some embodiments, the fat-related disorder is lipoma. In some embodiments, the fat-related disorder is fatty tumor disease. In some embodiments, the fat-related disorder is a disorder associated with fat accumulation. In some embodiments, the fat-related disorder is Dercum's disease. In some embodiments, the fat-related disorder is lipedema. In some embodiments, the fat-related disorder is hibernoma.

In some embodiments, the disease or disorder is fibrosis associated with a second disease or disorder. In some embodiments, the second disease or disorder is pulmonary fibrosis, liver cirrhosis, endomyocardial fibrosis, old myocardial infarction, atrial fibrosis, mediastinal fibrosis, myelofibrosis, retroperitoneal fibrosis, progressive massive fibrosis, nephrogenic systemic fibrosis, Carpal tunnel syndrome, joint fibrosis, Crohn's Disease, Keloid, Scleroderma, arthrofibrosis, Peyronic's disease, Dupuytren's contracture, or adhesive capsulitis.

In some embodiments, the disease or disorder is liposarcoma or a solid tumor. In some embodiments, the disease or disorder is liposarcoma. In some embodiments, the disease or disorder is a solid tumor.

In some embodiments, the disease or disorder is angiolipoma.

In another aspect, provided herein is use of the crystalline 5-(3,6-dibromo-9H-carbazol-9-yl)-N,N,N-trimethylpentan-1-aminium chloride, or hydrate thereof, provided herein or the pharmaceutical composition provided herein in the manufacture of a medicament for the treatment of a disease or disorder in a subject in need thereof.

In some embodiments, the disease or disorder is a fat-related disorder. In some embodiments, the fat-related disorder is obesity, abnormal fat distribution, diabetes, a cardiovascular disease, obstructive sleep apnea, lipoma, cancer, osteoarthritis, an endocrinologic disease, a reproductive disease, a neurological disease, a psychiatric disease, a rheumatological disease, or an orthopedic disease. In some embodiments, the fat-related disorder is cellulite, excess fat in various body areas, lipoma, fatty tumor disease, a disorder associated with fat accumulation, Dercum's disease, lipedema, or hibernoma. In some embodiments, the fat-related disorder is cellulite. In some embodiments, the fat-related disorder is excess fat in various body areas. In some embodiments, the fat-related disorder is lipoma. In some embodiments, the fat-related disorder is fatty tumor disease. In some embodiments, the fat-related disorder is a disorder associated with fat accumulation. In some embodiments, the fat-related disorder is Dercum's disease. In some embodiments, the fat-related disorder is lipedema. In some embodiments, the fat-related disorder is hibernoma.

In some embodiments, the disease or disorder is fibrosis associated with a second disease or disorder. In some embodiments, the second disease or disorder is pulmonary fibrosis, liver cirrhosis, endomyocardial fibrosis, old myocardial infarction, atrial fibrosis, mediastinal fibrosis, myelofibrosis, retroperitoneal fibrosis, progressive massive fibrosis, nephrogenic systemic fibrosis, Carpal tunnel syndrome, joint fibrosis, Crohn's Disease, Keloid, Scleroderma, arthrofibrosis, Peyronic's disease, Dupuytren's contracture, or adhesive capsulitis.

In some embodiments, the disease or disorder is liposarcoma or a solid tumor. In some embodiments, the disease or disorder is liposarcoma. In some embodiments, the disease or disorder is a solid tumor.

In some embodiments, the disease or disorder is angiolipoma.

As used herein, a therapeutically effective amount of crystalline Compound A refers to an amount sufficient to effect the intended application, including but not limited to, disease treatment, as defined herein. Also contemplated in the subject methods is the use of a sub-therapeutic amount of crystalline Compound A for treating an intended disease condition.

The amount of crystalline Compound A administered may vary depending upon the intended application (in vitro or in vivo), or the subject and disease condition being treated, e.g., the weight and age of the subject, the severity of the disease condition, the manner of administration and the like, which can readily be determined by one of ordinary skill in the art.

Measuring the biological effects of crystalline Compound A can comprise performing an assay on a biological sample, such as a sample from a subject. Any of a variety of samples may be selected, depending on the assay. Examples of samples include, but are not limited to, blood samples (e.g. blood plasma or serum), exhaled breath condensate samples, bronchoalveolar lavage fluid, sputum samples, urine samples, and tissue samples.

A subject being treated with crystalline Compound A may be monitored to determine the effectiveness of treatment, and the treatment regimen may be adjusted based on the subject's physiological response to treatment. For example, if inhibition of a biological effect is above or below a threshold, the dosing amount or frequency may be decreased or increased, respectively. The methods can further comprise continuing the therapy if the therapy is determined to be efficacious. The methods can comprise maintaining, tapering, reducing, or stopping the administered amount of a compound in the therapy if the therapy is determined to be efficacious. The methods can comprise increasing the administered amount of a compound in the therapy if it is determined not to be efficacious. Alternatively, the methods can comprise stopping therapy if it is determined not to be efficacious. In some embodiments, treatment with crystalline Compound A is discontinued if inhibition of the biological effect is above or below a threshold, such as in a lack of response or an adverse reaction. The biological effect may be a change in any of a variety of physiological indicators.

### V. COMPOSITIONS AND FORMULATIONS

The disclosure provides compositions, including pharmaceutical compositions, comprising a crystalline form or forms of the present invention.

In some embodiments, crystalline Compound A is formulated into pharmaceutical compositions. In specific embodiments, pharmaceutical compositions are formulated in a conventional manner using one or more physiologically acceptable carriers comprising excipients and auxiliaries which facilitate processing of the active compound/crystalline active compound into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company. 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising crystalline Compound A and a pharmaceutically acceptable diluent(s), excipient(s), or carrier(s). In certain embodiments, crystalline Compound A is administered as pharmaceutical compositions in which the crystalline Compound A is mixed with other active ingredients, as in combination therapy. Encompassed herein are all combinations of actives set forth in the combination therapies section below and throughout this disclosure. In specific embodiments, the pharmaceutical compositions include crystalline Compound A. In some embodiments, the crystalline Compound A is crystalline Form 1. In some embodiments, the crystalline Compound A is crystalline Form 2. In some embodiments, the crystalline Compound A is crystalline Form 3. In some embodiments, the crystalline Compound A is crystalline Form 4. In some embodiments, the crystalline Compound A is a mixture of crystalline Form 1 and crystalline Form 2. In some embodiments, the crystalline Compound A is a mixture of crystalline Form 1 and crystalline Form 3. In some embodiments, the crystalline Compound A is a mixture of crystalline Form 1 and crystalline Form 4. In some embodiments, the crystalline Compound A is a mixture of crystalline Form 2 and crystalline Form 3. In some embodiments, the crystalline Compound A is a mixture of crystalline Form 2 and crystalline Form 4. In some embodiments, the crystalline Compound A is a mixture of crystalline Form 3 and crystalline Form 4. In some embodiments, the crystalline Compound A is a mixture of crystalline Form 1, crystalline Form 2, and crystalline Form 3. In some embodiments, the crystalline Compound A is a mixture of crystalline Form 1, crystalline Form 2, and crystalline Form 4. In some embodiments, the crystalline Compound A is a mixture of crystalline Form 1, crystalline Form 3, and crystalline Form 4. In some embodiments, the crystalline Compound A is a mixture of crystalline Form 2, crystalline Form 3, and crystalline Form 4. In some embodiments, the crystalline Compound A is a mixture of crystalline Form 1, crystalline Form 2, crystalline Form 3, and crystalline Form 4.

A pharmaceutical composition, as used herein, refers to a mixture of crystalline Compound A with other chemical components, such as carriers, stabilizers, diluents, dispersing agents, suspending agents, thickening agents, and/or excipients. In certain embodiments, the pharmaceutical composition facilitates administration of the crystalline Compound A to an organism. In some embodiments, in practicing the methods of treatment or use provided herein, therapeutically effective amounts of crystalline Compound A are administered in a pharmaceutical composition to a mammal having a disease or condition to be treated. In specific embodiments, the mammal is a human. In certain embodiments, therapeutically effective amounts vary depending on the severity of the disease, the age and relative health of the subject and other factors. The crystalline Compound A described herein is used singly or in combination with one or more therapeutic agents as components of mixtures.

In one embodiment, crystalline Compound A is formulated in an aqueous solution. In specific embodiments, the aqueous solution is selected from, by way of example only, a physiologically compatible buffer, such as Hank's solution, Ringer's solution, or physiological saline buffer. In other embodiments, crystalline Compound A is formulated for transmucosal administration. In specific embodiments, transmucosal formulations include penetrants that are appropriate to the barrier to be permeated. In still other embodiments wherein the crystalline Compound A described herein is formulated for other parenteral injections, appropriate formulations include aqueous or nonaqueous solutions. In specific embodiments, such solutions include physiologically compatible buffers and/or excipients. In some embodiments, the aqueous solutions comprise propylene glycol. In some embodiments, the aqueous solutions comprise water and 5-95% propylene glycol (v/v %). In some embodiments, the aqueous solutions comprise water and 10-90% propylene glycol (v/v %). In some embodiments, the aqueous solutions comprise water comprise 20-80% propylene glycol (v/v %). In some embodiments, the aqueous solutions comprise water and 40-70% propylene glycol (v/v %). In some embodiments, the aqueous solutions comprise water and 55-65% propylene glycol (v/v %). In some embodiments, the aqueous solutions comprise water and 60% propylene glycol (v/v %).

In another embodiment, the crystalline Compound A described herein is formulated for oral administration. The crystalline Compound A is formulated by combining the crystalline Compound A with, e.g., pharmaceutically acceptable carriers or excipients. In various embodiments, the crystalline Compound A described herein is formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with crystalline Compound A described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, a therapeutically effective amount of crystalline Compound A described herein is formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In other embodiments, a therapeutically effective amount of crystalline Compound A described herein is formulated for buccal or sublingual administration. Formulations suitable for buccal or sublingual administration include, by way of example only, tablets, lozenges, or gels. In still other embodiments, the crystalline Compound A described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical composition of crystalline Compound A is formulated in a form suitable for parenteral injection as sterile suspension, solution or emulsion in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active crystalline Compound A in water-soluble form. In some embodiments, pharmaceutical formulations for parenteral administration comprise propylene glycol. In some embodiments, pharmaceutical formulations for parenteral administration comprise propylene glycol and water. In additional embodiments, suspensions of the active crystalline Compound A are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the crystalline Compound A to allow for the preparation of highly concentrated solutions. Alternatively, in other embodiments, the active ingredient is in powder form for constitution with a suitable vehicle, e.g., sterile pyrogen-free water, before use.

In still other embodiments, crystalline Compound A is administered topically. The crystalline Compound A described herein is formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

In yet other embodiments, crystalline Compound A is formulated for transdermal administration. In specific embodiments, transdermal formulations employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the transdermal delivery of the crystalline Compound A is accomplished by means of iontophoretic patches and the like. In certain embodiments, transdermal patches provide controlled delivery of the crystalline Compound A. In specific embodiments, the rate of absorption is slowed by using rate-controlling membranes or by trapping the compound within a polymer matrix or gel. In alternative embodiments, absorption enhancers are used to increase absorption. Absorption enhancers or carriers include absorbable pharmaceutically acceptable solvents that assist passage through the skin. For example, in one embodiment, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In other embodiments, crystalline Compound A is formulated for administration by inhalation. Various forms suitable for administration by inhalation include, but are not limited to, aerosols, mists or powders. Pharmaceutical compositions of the crystalline Compound A are conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules and cartridges of, such as, by way of example only, gelatin for use in an inhaler or insufflator are formulated containing a powder mix of the compound and a suitable powder base such as lactose or starch.

In still other embodiments, crystalline Compound A is formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In certain embodiments, pharmaceutical compositions are formulated in any conventional manner using one or more physiologically acceptable carriers comprising excipients and auxiliaries which facilitate processing of the active crystalline Compound A into preparations which can be used pharmaceutically. Proper formulation is dependent upon the route of administration chosen. Any pharmaceutically acceptable techniques, carriers, and excipients are optionally used as suitable. Pharmaceutical compositions comprising crystalline Compound A are manufactured in a conventional manner, such as, by way of example only, by means of conventional mixing, dissolving, granulating, dragee-making, levigating, emulsifying, encapsulating, entrapping or compression processes.

Pharmaceutical compositions include at least one pharmaceutically acceptable carrier, diluent or excipient and crystalline Compound A described herein as an active ingredient. The active ingredient is in free-acid or free-base form, or in a pharmaceutically acceptable salt form. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions, comprising crystalline Compound A described herein include formulating the crystalline Compound A with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, a pharmaceutical composition comprising crystalline Compound A illustratively takes the form of a liquid where the agents are present in solution, in suspension or both. Typically when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, useful aqueous suspension contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful pharmaceutical compositions also, optionally, include solubilizing agents to aid in the solubility of crystalline Compound A. The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, useful pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, useful compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other useful pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other useful compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other useful compositions include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methylpyrrolidone are also employed. In additional embodiments, the crystalline forms described herein are delivered using a sustained-release system, such as semipermeable matrices of solid hydrophobic polymers containing the therapeutic agent. Various sustained-release materials are useful herein. In some embodiments, sustained-release capsules release the crystalline form for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v, polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

### VI. ROUTES OF ADMINISTRATION

Suitable routes of administration include, but are not limited to, oral, intravenous, rectal, aerosol, parenteral, subcutaneous, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, crystalline Compound A is administered in a local rather than systemic manner, for example, via injection of the crystalline Compound A directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ-specific antibody. In such embodiments, the liposomes are targeted to and taken up selectively by the organ. In yet other embodiments, crystalline Compound A is provided in the form of a rapid release formulation, in the form of an extended release formulation, or in the form of an intermediate release formulation. In yet other embodiments, crystalline Compound A is administered topically. In some embodiments, crystalline Compound A is administered subcutaneously.

### VII. KITS/ARTICLES OF MANUFACTURE

For use in the therapeutic applications described herein, kits and articles of manufacture are also provided. In some embodiments, such kits comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers are formed from a variety of materials such as glass or plastic.

The articles of manufacture provided herein contain packaging materials. Packaging materials for use in packaging pharmaceutical products include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes the crystalline Compound A described herein, optionally in a composition or in combination with another agent as disclosed herein. The container(s) optionally have a sterile access port (for example the container is an intravenous solution bag or a vial having a stopper pierceable by a hypodermic injection needle). Such kits optionally comprising a compound with an identifying description or label or instructions relating to its use in the methods described herein.

The kits described herein may comprise one or more of the crystalline forms provided in this disclosure and a pharmaceutically acceptable excipient, carrier, diluent, solution, or buffer for reconstitution of the crystalline form to a drug product suitable for administration to an individual. The diluent, solution, or buffer may comprise, for example, water for injection, normal saline, or another suitable isotonic buffered solution. In some embodiments, the pharmaceutically acceptable excipient, carrier, diluent, solution, or buffer may comprise one or more of propylene glycol, polyoxyl hydrogenated castor oil, ethanol, glycerol, sorbitol, mannitol, benzyl alcohol, lauryl glucoside, ammonium lauryl sulfate, sodium lauryl sulfate, sodium laureth sulfate, sodium myreth sulfate, docusate, perfluorooetanesulfonate, perfluorobutanesulfonate, sodium stearate, sodium lauroyl sarcosinate, perfluorononanoate, perfluorooctanoate, cetrimonium bromide, cetylpyridinium chloride, benzalkonium chloride, benzethonium chloride, dimethyldioctadecylammonium chloride, dimethyldioctadecylammonium bromide, 3-[(3-cholamidopropyl)dimethylammonio]-1-propanesulfonate, cocamidopropyl hydroxysultaine, cocamidopropyl betaine, phosphatidylserine, phosphatidylethanolamine, phosphatidylcholine, sphingomyelin, octaethylene glycol monododecyl ether, pentaethylene glycol monododecyl ether, nonaethylene glycol, polyethylene glycol nonyl phenyl ether, 2-[4-(2,4,4-trimethylpentan-2-yl)phenoxy]ethanol, Polyoxyl-35 castor oil, polyethoxylated tallow amine, cocamide monoethanolamine, cocamide diethanolamine, glycerol monostearate, glycerol distearate, glycerol tristearate, glycerol monolaurate, glycerol dilaurate, glycerol trilaurate, sorbitan monolaurate, sorbitan monostearate, sorbitan tristearate, Polysorbate 20, Polysorbate 40, Polysorbate 60, Polysorbate 80, decyl glucoside, lauryl glucoside, octyl glucoside, lauryldimethylamine oxide, propionic acid, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneisosylic acid, behenic acid, tricosylic acid, lignoceric acid, pentacosylic acid, cerotic acid, carboceric acid, montanic acid, nonacosylic acid, melissic acid, hentriacontylic acid, lacceroic acid, psyllic acid, geddic acid, ceroplastic acid, hexatriacontylic acid, heptatriacontylic acid, octatriacontylic acid, nonatriacontylic acid, tetratriacontylic acid, crotonic acid, myristoleic acid, palmitoleic acid, sapienic acid, oleic acid, elaidic acid, vaccenic acid, gadoleic acid, eicosenoic acid, erucic acid, nervonic acid, linoleic acid, eicosadienoic acid, docosadienoic acid, linolenic acid, pinolenic acid, eleostearic acid, mead acid, dihomo-γ-linolenic acid, eicosatrienoic acid, stearidonic acid, arachidonic acid, eicosatetraenoic acid, adrenic acid, bosseopentaenoic acid, eicosapentaenoic acid, ozubondo acid, sardine acid, tetracosanolpentaenoic acid, cervonic acid, herring acid, arginine, histidine, lysine, aspartic acid, glutamic acid, serine, threonine, asparagine, glutamine, cysteine, glycine, proline, alanine, valine, isoleucine, leucine, methionine, phenylalanine, tyrosine, tryptophan, Vitamin E, ascorbyl palmitate, butylated hydroxytoluene, triethyl citrate, citric acid, or combinations thereof. In some embodiments, the pharmaceutically acceptable excipient, carrier, diluent, solution, or buffer may comprise one or more of propylene glycol, polyoxyl hydrogenated castor oil, ethanol, glycerol, sorbitol, mannitol, benzyl alcohol, lauryl glucoside, ammonium lauryl sulfate, sodium lauryl sulfate, sodium laureth sulfate, sodium myreth sulfate, sodium stearate, sodium lauroyl sarcosinate, benzalkonium chloride, benzethonium chloride, phosphatidylcholine, sphingomyelin, 2-[4-(2,4,4-trimethylpentan-2-yl)phenoxy]ethanol. Polyoxyl-35 castor oil, glycerol monostearate, glycerol distearate, glycerol tristearate, glycerol monolaurate, glycerol dilaurate, glycerol trilaurate, sorbitan monolaurate, sorbitan monostearate, sorbitan tristearate, Polysorbate 20, Polysorbate 40, Polysorbate 60, Polysorbate 80, lauric acid, tridecylic acid, myristic acid, palmitic acid, stearic acid, myristoleic acid, palmitoleic acid, sapienic acid, oleic acid, elaidic acid, vaccenic acid, gadoleic acid, arginine, histidine, lysine, aspartic acid, glutamic acid, serine, threonine, asparagine, glutamine, cysteine, glycine, proline, alanine, valine, isoleucine, leucine, methionine, phenylalanine, tyrosine, tryptophan, Vitamin E, ascorbyl palmitate, butylated hydroxytoluene, triethyl citrate, citric acid, or combinations thereof. In some embodiments, the pharmaceutically acceptable excipient, carrier, diluent, solution, or buffer may comprise one or more of ethanol, glycerol, propylene glycol, sorbitol, mannitol, benzyl alcohol, Polysorbate 20, Polysorbate 40, Polysorbate 60, Polysorbate 80, or combinations thereof. In some embodiments, the pharmaceutically acceptable excipient, carrier, diluent, solution, or buffer may comprise polysorbate 80, propylene glycol, benzyl alcohol, and water for injection. The pharmaceutically acceptable excipient, carrier, diluent, solution, or buffer may be provided in the same or separate containers. In some embodiments, the pharmaceutically acceptable excipient, carrier, diluent, solution, or buffer may be provided in the same container. In some embodiments, the pharmaceutically acceptable excipient, carrier, diluent, solution, or buffer may be provided in separate containers.

For example, a kit typically includes one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label is optionally on or associated with the container. For example, a label is on a container when letters, numbers or other characters forming the label are attached, molded or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical composition is presented in a pack or dispenser device which contains one or more unit dosage forms containing a compound provided herein. The pack for example contains metal or plastic foil, such as a blister pack. Or, the pack or dispenser device is accompanied by instructions for administration. Or, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing crystalline Compound A formulated in a compatible pharmaceutical carrier are prepared, placed in an appropriate container, and labeled for treatment of an indicated condition.

In some embodiments, disclosed herein is a kit comprising crystalline 5-(3,6-dibromo-9H-carbazol-9-yl)-N,N,N-trimethylpentan-1-aminium chloride (Compound A) and one or more pharmaceutically acceptable excipients. In some embodiments, the crystalline 5-(3,6-dibromo-9H-carbazol-9-yl)-N,N,N-trimethylpentan-1-aminium chloride (Compound A) is anhydrous. In some embodiments, the crystalline 5-(3,6-dibromo-9H-carbazol-9-yl)-N,N,N-trimethylpentan-1-aminium chloride (Compound A) is hydrated. In some embodiments, crystalline Compound A is crystalline Compound A Form 1. In some embodiments, crystalline Compound A is crystalline Compound A Form 2. In some embodiments, crystalline Compound A is crystalline Compound A Form 3. In some embodiments, crystalline Compound A is crystalline Compound A Form 4. In some embodiments, Compound A is Compound A Form 1. In some embodiments, the crystalline Compound A is a mixture of crystalline Form 1 and crystalline Form 2. In some embodiments, the crystalline Compound A is a mixture of crystalline Form 1 and crystalline Form 3. In some embodiments, the crystalline Compound A is a mixture of crystalline Form 1 and crystalline Form 4. In some embodiments, the crystalline Compound A is a mixture of crystalline Form 2 and crystalline Form 3. In some embodiments, the crystalline Compound A is a mixture of crystalline Form 2 and crystalline Form 4. In some embodiments, the crystalline Compound A is a mixture of crystalline Form 3 and crystalline Form 4. In some embodiments, the crystalline Compound A is a mixture of crystalline Form 1, crystalline Form 2, and crystalline Form 3. In some embodiments, the crystalline Compound A is a mixture of crystalline Form 1, crystalline Form 2, and crystalline Form 4. In some embodiments, the crystalline Compound A is a mixture of crystalline Form 1, crystalline Form 3, and crystalline Form 4. In some embodiments, the crystalline Compound A is a mixture of crystalline Form 2, crystalline Form 3, and crystalline Form 4. In some embodiments, the crystalline Compound A is a mixture of crystalline Form 1, crystalline Form 2, crystalline Form 3, and crystalline Form 4.

In some embodiments, provided herein is a kit comprising crystalline 5-(3,6-dibromo-9H-carbazol-9-yl)-N,N,N-trimethylpentan-1-aminium chloride (Compound A) and one or more pharmaceutically acceptable excipients for use in the treatment of a disease or disorder. In some embodiments, the disease or disorder is a fat-related disease or disorder, a fibrosis related disease or disorder, a liposarcoma, a solid tumor, or an angiolipoma. In some embodiments, the disease or disorder is a fat-related disorder. In some embodiments, the fat-related disorder is obesity, abnormal fat distribution, diabetes, a cardiovascular disease, obstructive sleep apnea, lipoma, cancer, osteoarthritis, an endocrinologic disease, a reproductive disease, a neurological disease, a psychiatric disease, a rheumatological disease, or an orthopedic disease. In some embodiments, the fat-related disorder is cellulite, excess fat in various body areas, lipoma, fatty tumor disease, a disorder associated with fat accumulation, Dercum's disease, lipedema, or hibernoma. In some embodiments, the fat-related disorder is cellulite. In some embodiments, the fat-related disorder is excess fat in various body areas. In some embodiments, the fat-related disorder is lipoma. In some embodiments, the fat-related disorder is fatty tumor disease. In some embodiments, the fat-related disorder is a disorder associated with fat accumulation. In some embodiments, the fat-related disorder is Dercum's disease. In some embodiments, the fat-related disorder is lipedema. In some embodiments, the fat-related disorder is hibernoma. In some embodiments, the disease or disorder is fibrosis associated with a second disease or disorder. In some embodiments, the second disease or disorder is pulmonary fibrosis, liver cirrhosis, endomyocardial fibrosis, old myocardial infarction, atrial fibrosis, mediastinal fibrosis, myelofibrosis, retroperitoneal fibrosis, progressive massive fibrosis, nephrogenic systemic fibrosis, Carpal tunnel syndrome, joint fibrosis, Crohn's Disease, Keloid, Scleroderma, arthrofibrosis, Peyronie's disease. Dupuytren's contracture, or adhesive capsulitis. In some embodiments, the disease or disorder is liposarcoma or a solid tumor. In some embodiments, the disease or disorder is liposarcoma. In some embodiments, the disease or disorder is a solid tumor. In some embodiments, the disease or disorder is angiolipoma.

### EXAMPLES

The following examples serve to more fully describe the manner of using the invention. These examples are presented for illustrative purposes and should not serve to limit the true scope of the invention.

In some embodiments, 5-(3,6-dibromo-9H-carbazol-9-yl)-N,N,N-trimethylpentan-1-aminium chloride (Compound A) is prepared as described in WO 2013/072915 and U.S. Pat. No. 9,447,040, which is incorporated by reference for such synthesis. In some embodiments, Compound A is prepared as described herein.

### Example 1: Preparation of 5-(3,6-dibromo-9H-carbazol-9-yl)-N,N,N-trimethylpentan-1-aminium chloride (Compound A)

3,6-dibromocarbazole (1.00 g, 3.0 mM) was dissolved in dimethylformamide (100 mL). (5-bromopentyl)-trimethyl-ammonium bromide (0.57 g, 3.0 mM) was added at once. After 10 minutes of magnetic stirring, potassium carbonate (1.40 g, 10 mM) was added. After an additional 10 minutes of stirring, the temperature was raised to 50 °C and the mixture was stirred for 4 hours. After cooling to room temperature, the solution was transferred to a separatory funnel and water (200 mL) and dichloromethane (200 mL) were added. The solvent mixture was shaken, and the lower phase was collected. The upper aqueous phase was extracted four times with 3:1 dichloromethane/methanol (50 mL). The combined organics were washed with brine (100 mL), dried over magnesium sulfate, filtered, and concentrated. The resulting residue was crystallized from water to afford 5-(3,6-dibromo-9H-carbazol-9-yl)-N,N,N-trimethylpentan-1-aminium chloride (Compound A) (1.28 g). ¹H NMR (CD₃OD, 400 MHz) δ 8.21 (d, J=0.4 Hz, 2H), 7.54 (dd, J=2.0, 0.4 Hz, 2H), 7.47 (d, J=2.0, 2H), 4.39 (t. J=0.6, 2H), 3.16 (m, 2H), 3.01 (s, 9H), 1.93 (m, 2H), 1.70 (m, 2H), 1.31 (m, 2H). MS: m/z=451, 453, 455 (M⁺, symmetrical 2Br triplet) and 452, 454, 456 (M+H⁺, symmetrical 2Br triplet).

### Example 2: Temperature Cycling Preparation of Crystalline 5-(3,6-dibromo-9H-carbazol-9-yl)-N,N,N-trimethylpentan-1-aminium chloride Form 1 (Compound A Form 1)

To a known mass of 5-(3,6-dibromo-9H-carbazol-9-yl)-N,N,N-trimethylpentan-1-aminium chloride (Compound A) was added a solvent (selected from 1-butanol, 2-propanol, acetone, acetonitrile, benzyl alcohol, dichloromethane, dimethylsulfoxide, ethanol, ethyl acetate, isopropyl acetate, methanol, methyl ethyl ketone, propylene glycol, methyl isobutyl ketone, tert-butyl methyl ether, tetrahydrofuran, toluene. Tween 80, water, tert-butyl methyl ether/water (2:98 v/v %), acetonitrile/1-butanol/tert-butyl methyl ether/water (2:2:2:94 v/v %), acetonitrile/l-butanol/tert-butyl methyl ether (25:25:50 v/v %), Tween 80/propylene glycol/benzyl alcohol/water (10:60:3:27 w/w %), Tween 80/propylene glycol/benzyl alcohol (14:81:5 w/w %), methanol/water (40:60 v/v %), and methanol/water (80:00 v/v %)) in 0.5 mL aliquots until a mobile slurry was observed. The reaction mixtures were maintained at 50 °C with stirring during the solvent addition and then temperature cycled between 5 °C and 50 °C with 1 hour hold periods and at the low and elevated temperatures. After cycling for 18 hours, tert-butyl methyl ether was added if the reaction mixture remained a clear solution; addition was carried out until a slurry was observed or a total volume of 1.8 mL was reached. The reaction mixtures were then left to continue temperature cycling for a total of 72 hours. The saturated solutions were extracted, needle-filtered, and stored at ambient temperature for further screening experiments. The resulting solids were dried under vacuum at ambient temperature for 3 days. The 1-butanol, 2-propanol, acetone, acetonitrile, benzyl alcohol, dichloromethane, dimethylsulfoxide, ethanol, ethyl acetate, isopropyl acetate, methanol, methyl ethyl ketone, methyl isobutyl ketone, tert-butyl methyl ether, tetrahydrofuran, toluene, Tween 80, tert-butyl methyl ether/water (2:98 v/v %), acetonitrile/1-butanol/tert-butyl methyl ether (25:25:50 v/v %), Tween 80/propylene glycol/benzyl alcohol (14:81:5 w/w %), and methanol/water (40:60 v/v %) solutions afforded crystalline Compound A (Form 1).

### Example 3: Crash Cooling Preparation of Crystalline 5-(3,6-dibromo-9H-carbazol-9-yl)-N,N,N-trimethylpentan-1-aminium chloride Form 1 (Compound A Form 1)

The solutions obtained in Example 2 were rapidly cooled to 6 °C. After 4 days, the ethanol solution afforded crystalline Compound A. The rest of the solutions were rapidly cooled to −18 °C. After 48 hours, the 1-butanol, 2-propanol, acetonitrile, Tween 80/propylene glycol/benzyl alcohol (14:81:5 w/w %), methanol/water (40:60 v/v %), and methanol/water (80:20 v/v %) solutions afforded crystalline Form 1 of Compound A. The remaining solutions were treated with tert-butyl methyl ether. After 18 hours, the benzyl alcohol and dimethylsulfoxide solutions afforded crystalline Compound A (Form 1).

### Example 4: Evaporative Preparation of Crystalline 5-(3,6-dibromo-9H-carbazol-9-yl)-N,N,N-trimethylpentan-1-aminium chloride Form 1 (Compound A Form 1)

The solutions obtained in Example 2 were added to open vials at room temperature. After 4 days, the 1-butanol, 2-propanol, acetonitrile, methanol, acetonitrile/1-butanol/tert-butyl methyl ether/water (2:2:2:94 v/v %), acetonitrile/1-butanol/tert-butyl methyl ether (25:25:50 v/v %), Tween 80/propylene glycol/benzyl alcohol (14:81:5 w/w %), methanol/water (40:60 v/v %), and methanol/water (80:00 v/v %) solutions afforded crystalline Compound A (Form 1).

### Example 5: Anti-Solvent Addition Preparation of Crystalline 5-(3,6-dibromo-9H-carbazol-9-yl)-N,N,N-trimethylpentan-1-aminium chloride Form 1 (Compound A Form 1)

The solutions obtained in Example 2 were added to vials and tert-butyl methyl ether was added in 50 μL aliquots at ambient temperature until precipitation was observed or a total of 1.8 mL of anti-solvent had been added. Solids were isolated by centrifugation. The remaining solutions were cooled to 6 °C, stored for 72 hours, cooled to −18 °C, and stored for 48 hours. The 1-butanol, benzyl alcohol, dimethylsulfoxide, ethanol, methanol, acetonitrile/1-butanol/tert-butyl methyl ether (25:25:50 v/v %), and methanol/water (80:00 v/v %) solutions afforded crystalline Compound A (Form 1).

### Example 6: Preparation of Crystalline 5-(3,6-dibromo-9H-carbazol-9-yl)-N,N,N-trimethylpentan-1-aminium chloride Form 2 (Compound A Form 2) from Compound A Form 1

A known mass of 5-(3,6-dibromo-9H-carbazol-9-yl)-N,N,N-trimethylpentan-1-aminium chloride Form 1 (Compound A Form 1) was added into a pre-tared open aluminum pan and loaded into a TA Instruments Discovery SDT 650 Auto—Simultaneous DSC and held at room temperature. The sample was then heated at a rate of 10 °C per minute from 30 °C to 200 °C. The sample was held at 200 °C for 10 minutes before being allowed to cool to 30 °C. The process afforded crystalline Compound A (Form 2).

### Example 7: Preparation of Crystalline 5-(3,6-dibromo-9H-carbazol-9-yl)-N,N,N-trimethylpentan-1-aminium chloride Form 3 (Compound A Form 3)

### Synthesis

3,6-dibromocarbazole was dissolved in MeCN/MeTHF. (5-bromopentyl)-trimethyl-ammonium bromide was added at once. After 10 minutes of magnetic stirring, potassium carbonate was added. After an additional 10 minutes of stirring, the temperature was raised to 50 °C and the mixture was stirred for 4 hours. After cooling to room temperature, the solution was transferred to a separatory funnel and water and dichloromethane were added. The solvent mixture was shaken, and the lower phase was collected. The upper aqueous phase was extracted four times with 3:1 dichloromethane/methanol. The combined organics were washed with brine, dried over magnesium sulfate, filtered, and concentrated. A portion of the resulting 5-(3,6-dibromo-9H-carbazol-9-yl)-N,N,N-trimethylpentan-1-aminium chloride (Compound A) was subjected to bead milling (6 cycles for 60 seconds each at 6000 rpm with a 10 second rest between cycles). Milled and non-milled samples were placed in crystallizing dishes, protected from dust, and exposed to air at ambient temperatures for 15 days at a relative humidity of 20-25%. Both milled and non-milled samples afforded crystalline Compound A (Form 3).

### Temperature Cycling

To a known mass of 5-(3,6-dibromo-9H-carbazol-9-yl)-N,N,N-trimethylpentan-1-aminium chloride (Compound A) was added water until a mobile slurry was observed. The slurry was heated gradually until compound dissolution at about 56 °C. The solution was further heated to 85 °C, cooled quickly to 65 °C, then cooled slowly to 20 °C over 9 hours. The slurry was stirred at 140 RPM for 7 days. A lower stirring rate of 110 RPM was implemented for two additional heat/cool cycles. The slurry was reheated to about 56 °C where a very thin slurry was formed, aged for 1 hour, and cooled to 20 °C over 8 hours. The slurry was then allowed to cool for 5 hours at 20 °C when the solid was isolated via filtration with a polypropylene felt filter (2 microns) to yield a wet cake. The cake was rinsed with water, washed twice with MTBE, and dried under vacuum for 1 hour. Drying was continued for an additional 1 hour to afford crystalline Compound A (Form 3).

### Example 8: Preparation of Crystalline 5-(3,6-dibromo-9H-carbazol-9-yl)-N,N,N-trimethylpentan-1-aminium chloride Form 4 (Compound A Form 4) from Compound A

A known mass of 5-(3,6-dibromo-9H-carbazol-9-yl)-N,N,N-trimethylpentan-1-aminium chloride was dried until water content reached ≤1% (w/w). The dried sample was held in conditions with low humidity. The process afforded crystalline Compound A (Form 4).

### Example 9: Preparation of Crystalline 5-(3,6-dibromo-9H-carbazol-9-yl)-N,N,N-trimethylpentan-1-aminium chloride Form 1 (Compound A Form 1) from Compound A Form 2

A known mass of 5-(3,6-dibromo-9H-carbazol-9-yl)-N,N,N-trimethylpentan-1-aminium chloride Form 2 (Compound A Form 2) was analyzed by variable humidity X-ray powder diffraction (VH-XRPD) on a Philips X'Pert Pro Multipurpose diffractometer quipped with a humidity chamber. The samples were scanned between 4 and 35.99° 2θ using Cu K radiation (α1 λ=1.54060 Å; α2=1.54443 Å; β=1.39225 Å; α1:α2 ratio=0.5) running in Bragg-Brentano geometry (step size 0.008° 2θ) using 40 kV/40 mA generator settings. Measurements were performed between 7-90% RH and XRPD scans were collected at each humidity step. The VH-XRPD analysis was conducted an absorption phase between ambient and 90% RH, a desorption phase between 90% RH and 7% RH, and a second absorption phase between 20% and 40% RH. Crystalline Form 1 of Compound A was observed during the desorption phase between 90% RH and 20% RH, and in the second absorption phase at 40% RH.

### Example 10: Preparation of Crystalline 5-(3,6-dibromo-9H-carbazol-9-yl)-N,N,N-trimethylpentan-1-aminium chloride Form 1 (Compound A Form 1) from Compound A Form 4

A known mass of 5-(3,6-dibromo-9H-carbazol-9-yl)-N,N,N-trimethylpentan-1-aminium chloride Form 4 (Compound A Form 4) was converted to crystalline Form 1 of Compound A after standing at 25 °C and 40 °C and at >30% RH.

### Example 11: X-Ray Powder Diffraction (XRPD)

Although the following diffractometers were used, other types of diffractometers could be used. Furthermore, other wavelengths could be used and converted to the Cu Kα. In some embodiments, Synchrotron Radiation X-Ray Powder Diffraction (SR-XRPD) can be used to characterize the crystalline forms.

“Characteristic peaks”, to the extent they exist, are a subset of observed peaks and are used to differentiate one crystalline polymorph from another crystalline polymorph (polymorphs being crystalline forms having the same chemical composition). Characteristic peaks are determined by evaluating which observed peaks, if any, are present in one crystalline polymorph of a compound against all other known crystalline polymorphs of that compound to within ±0.2° 2-Theta.

XRPD analysis was carried out on a PANalytical X'pert Pro with PIXcel detector (128 channels), scanning the samples between 3 and 35° 20. The material was gently ground (where required) to release any agglomerates and loaded onto a multi-well plate with Kapton or Mylar polymer film to support the sample. The multi-well plate was then placed into the diffractometer and analyzed using Cu K radiation (α₁ λ=1.54060 Å; α₂ λ=1.54443 Å; β λ=1.39225 Å; α₁:α₂ ratio=0.5) running in transmission mode (step size 0.0130° 2θ, step time 18.87 s) using 40 kV/40 mA generator settings. Data were visualized and images generated using the HighScore Plus 4.7 desktop application (PANalytical, 2017).

A representative XRPD pattern obtained for crystalline Compound A (Form 1) is displayed in FIG. 1. Representative XRPD peaks obtained for crystalline Compound A (Form 1) are listed in Table 1.

TABLE 1
W

[symbol]

| Peak # | Angle (° 2-θ) | Intensity (%) |
|---|---|---|
| 1 | 4.0 | 100 |
| 2 | 7.7 | 2.04 |
| 3 | 10.4 | 2.68 |
| 4 | 12.1 | 3.83 |
| 5 | 13.1 | 6.65 |
| 6 | 14.3 | 6.92 |
| 7 | 14.5 | 3.78 |
| 8 | 15.5 | 32.32 |
| 9 | 16.1 | 6.78 |
| 10 | 16.5 | 35.53 |
| 11 | 16.8 | 4.04 |
| 12 | 17.6 | 5.49 |
| 13 | 18.4 | 9.55 |
| 14 | 18.9 | 2.74 |
| 15 | 19.9 | 12.18 |
| 16 | 20.8 | 10.22 |
| 17 | 21.0 | 9.3 |
| 18 | 21.3 | 4.89 |
| 19 | 21.8 | 9.15 |
| 20 | 22.2 | 4.54 |
| 21 | 22.7 | 6.61 |
| 22 | 23.3 | 5.64 |
| 23 | 23.8 | 17.45 |
| 24 | 24.1 | 30.26 |
| 25 | 24.6 | 11.61 |
| 26 | 24.9 | 5.85 |
| 27 | 25.3 | 6.48 |
| 28 | 26.0 | 26.84 |
| 29 | 26.1 | 30.75 |
| 30 | 26.5 | 22.16 |
| 31 | 27.6 | 3.09 |
| 32 | 28.3 | 6.34 |
| 33 | 29.5 | 5.03 |
| 34 | 30.8 | 6.08 |
| 35 | 31.2 | 8.05 |
| 36 | 31.9 | 4.2 |
| 37 | 32.6 | 5.01 |
| 38 | 33.6 | 4.85 |
| 39 | 34.5 | 5.01 |

[symbol]

indicates data missing or illegible when filed

In some embodiments, representative XRPD peaks obtained for crystalline Compound A (Form 1) are listed in Table 1-1.

TABLE 1-1

| Angle (° 2-θ) | Intensity (%) |
|---|---|
| 4.0 | 100 |
| 15.4 | 32.32 |
| 16.4 | 35.53 |
| 19.9 | 12.18 |
| 23.8 | 17.45 |
| 24.1 | 30.26 |
| 24.5 | 11.61 |
| 26.0 | 26.84 |
| 26.0 | 30.75 |
| 26.4 | 22.16 |

A representative XRPD pattern obtained for the crystalline Compound A (Form 2) is displayed in FIG. 4. Representative XRPD peaks obtained for the crystalline Compound A (Form 2) are listed in Table 2.

TABLE 2

| Angle (° 2-θ) | Intensity (%) |
|---|---|
| 4.8 | 100 |
| 15.0 | 37.46 |
| 17.5 | 63.57 |
| 17.9 | 56.62 |
| 19.6 | 43.09 |
| 20.4 | 37.98 |
| 21.0 | 43.18 |
| 24.8 | 88.6 |
| 25.5 | 40.01 |
| 26.5 | 89.07 |

A representative XRPD pattern obtained for the crystalline Compound A (Form 3) is displayed in FIG. 7. Representative XRPD peaks obtained for the crystalline Compound A (Form 3) are listed in Table 3.

TABLE 3

| Angle (° 2-θ) | Intensity* |
|---|---|
| 12.1 | 9 |
| 18.5 | 8 |
| 20.7 | 14 |
| 21.8 | 17 |
| 23.3 | 13 |
| 26.8 | 11 |
| 28.4 | 6 |
| 30.8 | 16 |
| 31.7 | 12 |
| 34.5 | 18 |

*Where 1 is the peak with the most signal intensity and 30 is the peak with the least signal intensity.

A representative XRPD pattern obtained for the crystalline Compound A (Form 4) is displayed in FIG. 8. Representative XRPD peaks obtained for the crystalline Compound A (Form 4) are listed in Table 4.

TABLE 4

| Angle (° 2-θ) | Intensity * |
|---|---|
| 3.0 | 5 |
| 4.0 | 3 |
| 5.0 | 1 |
| 15.3 | 2 |
| 17.6 | 9 |
| 20.6 | 8 |
| 22.3 | 7 |
| 24.1 | 10 |
| 25.6 | 4 |
| 31.2 | 6 |

* Where of the representative peaks listed, 1 is the peak with the most signal intensity and 10 is the peak with the least signal intensity.

### Example 12: Thermogravimetric Analysis/Differential Scanning Calorimetry (TGA/DSC)

5-10 mg of material was added into a pre-tared open aluminum pan and loaded into a TA Instruments Discovery SDT 650 Auto—Simultaneous DSC and held at room temperature. The sample was then heated at a rate of 10 °C per minute from 30 °C to 400 °C during which time the change in sample weight was recorded along with the heat flow response (DSC). Nitrogen was used as the sample purge gas at a flow rate of 200 cm³ per minute. The TGA pattern obtained for crystalline Compound A (Form 1) is shown in FIG. 3 and the TGA pattern obtained for crystalline Compound A (Form 2) is shown in FIG. 6.

### Example 13: Differential Scanning Calorimetry (DSC)

1-5 mg of material was weighed into an aluminum DSC pan and seal non-hermetically with an aluminum lid. The sample pan was then loaded into a TA Instruments Discovery DSC 2500 differential scanning calorimeter equipped with a RC90 cooler. The sample and reference were heated to 300 °C at a scan rate of 10 °C per minute and the resulting heat flow response monitored. The sample was re-cooled to 20 °C and then reheated again to 300 °C all at 10 °C per minute. Nitrogen was used as the purge gas at a flow rate of 50 cm³ per minute. The DSC thermogram obtained for crystalline Compound A (Form 1) is shown in FIG. 2. The DSC thermogram obtained for crystalline compound A (Form 2) is shown in FIG. 5.

### Example 14: Stability Tests of Crystalline Compound A Form 1

Crystalline Compound A (Form 1) was not changed after storage at room temperature for 7 days, storage at 40 °C/75% RH for 7 days, or storage at 80 °C for 7 days. The results of crystalline Compound A (Form 1) in accelerated stability tests are shown in Table 5. Purity was determined by HPLC analysis.

TABLE 5

| Conditions | Time Point | Purity | XRPD |
|---|---|---|---|
| Room Temperature (open vial) | 7 days | 99.93% | Unchanged |
| 40 °C/75% RH (open vial) | 7 days | 99.93% | Unchanged |
| 80 °C (closed vial) | 7 days | 99.93% | Unchanged |

Crystalline Compound A (Form 1) was not changed after storage at 25 °C/60% RH for 36 months or storage at 40 °C/75% RH for 6 months. The results of crystalline Compound A (Form 1) in long-term stability tests are shown in Table 6. Purity was determined by HPLC analysis.

TABLE 6

| Conditions | Time Point | Purity | XRPD |
|---|---|---|---|
| 25 °C/60% RH (closed | 3 months | 99.7% | Unchanged |
| container) | | | |
| 25 °C/60% RH (closed | 6 months | 99.7% | Unchanged |
| container) | | | |
| 25 °C/60% RH (closed | 12 months | 99.7% | Unchanged |
| container) | | | |
| 25 °C/60% RH (closed | 24 months | 99.7% | Unchanged |
| container) | | | |
| 25 °C/60% RH (closed | 36 months | 99.7% | Unchanged |
| container) | | | |
| 40 °C/75% RH (closed | 1 month | 99.7% | Unchanged |
| container) | | | |
| 40 °C/75% RH (closed | 3 months | 99.7% | Unchanged |
| container) | | | |
| 40 °C/75% RH (closed | 6 months | 99.8% | Unchanged |
| container) | | | |

### Example 15: Karl Fischer (KF) Coulometric Titration

Water content of crystalline Compound A was determined using KF Coulometric Titration by either direct addition or liquid extraction.

### Direct Addition

10-20 mg of crystalline Compound A was weighed into a vial. The solid was then manually introduced into the titration cell of a Mettler Toledo C30 Compact Titrator. The vial was back-weighed after the addition of the solid and the weight of the added solid entered on the instrument. Titration was initiated once the sample had fully dissolved in the cell. The water content was calculated automatically by the instrument as a percentage and the data printed. The analysis was carried out in duplicate.

### Liquid Extraction

10-20 mg of crystalline Compound A was weighed into a vial, 10-15 mL of the Coulomat was extracted from the cell of a Mettler Toledo C30 Compact Titrator and added to the vial containing the sample. The solid was dissolved by shaking manually or using ultrasonic bath. When complete dissolution was achieved, the solution was injected back into the cell titrator and the weight of the sample added was entered on the instrument. The water content was calculated automatically by the instrument as a percentage and the data printed. The analysis was carried out in duplicate.

The KF titration results obtained for crystalline Compound A is summarized in Table 7.

TABLE 7

| | Water content | Water:Compound |
|---|---|---|
| Crystalline Form | (w/w %) | ratio |
| Form 1 | 4.38% | ~2:1 |
| Form 3 | 5.3% | ~1.5:1 |
| Form 4 | 1% | — |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Disclosure of the present application is further illustrated in the following list of embodiments, which are given for illustration purposes only and are not intended to limit the disclosure in any way:

Embodiment 1: Crystalline 5-(3,6-dibromo-9H-carbazol-9-yl)-N,N,N-trimethylpentan-1-aminium chloride (Compound A):

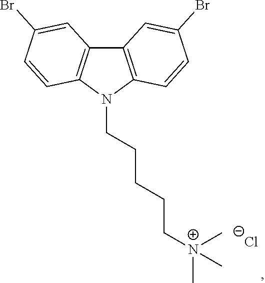 [SMILES: C[N+](C)(C)CCCCCn1c2ccc(Br)cc2c2cc(Br)ccc21 ; [Cl-]]

or hydrate thereof, wherein the crystalline 5-(3,6-dibromo-9H-carbazol-9-yl)-N,N,N-trimethylpentan-1-aminium chloride is characterized as having:
   (a) an X-ray powder diffraction pattern comprising peaks at 4.0±0.2° 2-θ, 16.5±0.2° 2-θ, and 15.5±0.2° 2-θ, as measured using Cu Kα1 radiation with an X-ray wavelength of 1.54060 Å;
   (b) an X-ray powder diffraction pattern substantially the same as shown in FIG. 1, as measured using Cu Kα1 radiation with an X-ray wavelength of 1.54060 Å;
   (c) a differential scanning calorimetry (DSC) thermogram comprising:
      i) an endotherm in the range of about 260-270 °C; and
      ii) an endotherm in the range of about 175-195 °C;
   (d) a differential scanning calorimetry (DSC) thermogram comprising:
      i) an endotherm with an onset of about 265 °C and a peak of about 266 °C; and
      ii) an endotherm with an onset of about 180 °C and a peak of about 190 °C;
   (e) a differential scanning calorimetry (DSC) thermogram substantially the same as shown in FIG. 2;
   (f) a Thermogravimetric Analysis (TGA) thermogram substantially the same as shown in FIG. 3;
   (g) an unchanged XRPD after storage at 40 °C and 75% relative humidity (RH) for 7 days;
   (h) an unchanged XRPD after storage at 80 °C for 7 days;
   or
   (i) combinations thereof.

Embodiment 2: The crystalline 5-(3,6-dibromo-9H-carbazol-9-yl)-N,N,N-trimethylpentan-1-aminium chloride of embodiment 1, or hydrate thereof, characterized as having an X-ray powder diffraction pattern comprising peaks at 4.0±0.2° 2-θ, 16.5±0.2° 2-θ, and 15.5±0.2° 2-θ, as measured using Cu Kα1 radiation with an X-ray wavelength of 1.54060 Å.

Embodiment 3: The crystalline 5-(3,6-dibromo-9H-carbazol-9-yl)-N,N,N-trimethylpentan-1-aminium chloride of embodiment 1 or 2, or hydrate thereof, wherein the crystalline 5-(3,6-dibromo-9H-carbazol-9-yl)-N,N,N-trimethylpentan-1-aminium chloride is characterized as having an X-ray powder diffraction pattern substantially the same as shown in FIG. 1, as measured using Cu Kα1 radiation with an X-ray wavelength of 1.54060 Å.

Embodiment 4: The crystalline 5-(3,6-dibromo-9H-carbazol-9-yl)-N,N,N-trimethylpentan-1-aminium chloride of any one of embodiments 1 to 3, or hydrate thereof, wherein the crystalline 5-(3,6-dibromo-9H-carbazol-9-yl)-N,N,N-trimethylpentan-1-aminium chloride is characterized by a differential scanning calorimetry (DSC) thermogram comprising:
i) an endotherm in the range of about 260-270 °C; and
ii) an endotherm in the range of about 175-195 °C.

Embodiment 5: The crystalline 5-(3,6-dibromo-9H-carbazol-9-yl)-N,N,N-trimethylpentan-1-aminium chloride of any one of embodiments 1 to 4, or hydrate thereof, wherein the crystalline 5-(3,6-dibromo-9H-carbazol-9-yl)-N,N,N-trimethylpentan-1-aminium chloride is characterized by a differential scanning calorimetry (DSC) thermogram comprising:
i) an endotherm with an onset of about 265 °C and a peak of about 266 °C; and
ii) an endotherm with an onset of about 180 °C and a peak of about 190 °C.

Embodiment 6: The crystalline 5-(3,6-dibromo-9H-carbazol-9-yl)-N,N,N-trimethylpentan-1-aminium chloride of any one of embodiments 1 to 5, or hydrate thereof, wherein the crystalline 5-(3,6-dibromo-9H-carbazol-9-yl)-N,N,N-trimethylpentan-1-aminium chloride is characterized by a differential scanning calorimetry (DSC) thermogram substantially the same as shown in FIG. 2.

Embodiment 7: The crystalline 5-(3,6-dibromo-9H-carbazol-9-yl)-N,N,N-trimethylpentan-1-aminium chloride of any one of embodiments 1 to 6, or hydrate thereof, wherein the crystalline 5-(3,6-dibromo-9H-carbazol-9-yl)-N,N,N-trimethylpentan-1-aminium chloride is characterized by a Thermogravimetric Analysis (TGA) thermogram substantially the same as shown in FIG. 3.

Embodiment 8: Crystalline 5-(3,6-dibromo-9H-carbazol-9-yl)-N,N,N-trimethylpentan-1-aminium chloride (Compound A):

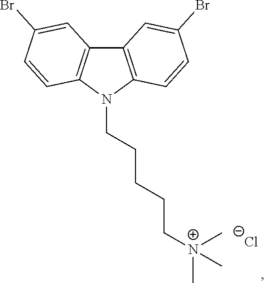 [SMILES: C[N+](C)(C)CCCCCn1c2ccc(Br)cc2c2cc(Br)ccc21 ; [Cl-]]

or hydrate thereof, wherein the crystalline 5-(3,6-dibromo-9H-carbazol-9-yl)-N,N,N-trimethylpentan-1-aminium chloride is characterized as having:
   (a) an X-ray powder diffraction pattern comprising peaks at 28.4±0.2° 2-θ, 18.5±0.2° 2-θ, and 12.1±0.2° 2-θ, as measured using Cu Kα1 radiation with an X-ray wavelength of 1.54060 Å;
   (b) an X-ray powder diffraction pattern substantially the same as shown in FIG. 7, as measured using Cu Kα1 radiation with an X-ray wavelength of 1.54060 Å;
   or
   (c) combinations thereof.

Embodiment 9: The crystalline 5-(3,6-dibromo-9H-carbazol-9-yl)-N,N,N-trimethylpentan-1-aminium chloride of embodiment 8, or hydrate thereof, wherein the crystalline 5-(3,6-dibromo-9H-carbazol-9-yl)-N,N,N-trimethylpentan-1-aminium chloride is characterized as having an X-ray powder diffraction pattern comprising peaks at 28.4±0.2° 2-θ, 18.5±0.2° 2-θ, and 12.1±0.2° 2-θ, as measured using Cu Kα1 radiation with an X-ray wavelength of 1.54060 Å.

Embodiment 10: The crystalline 5-(3,6-dibromo-9H-carbazol-9-yl)-N,N,N-trimethylpentan-1-aminium chloride of embodiment 8 or 9, or hydrate thereof, wherein the crystalline 5-(3,6-dibromo-9H-carbazol-9-yl)-N,N,N-trimethylpentan-1-aminium chloride is characterized as having an X-ray powder diffraction pattern substantially the same as shown in FIG. 7, as measured using Cu Kα1 radiation with an X-ray wavelength of 1.54060 Å.

Embodiment 11: A pharmaceutical composition comprising the crystalline 5-(3,6-dibromo-9H-carbazol-9-yl)-N,N,N-trimethylpentan-1-aminium chloride, or hydrate thereof, of any one of embodiments 1 to 10, and at least one pharmaceutically acceptable excipient.

Embodiment 12: The pharmaceutical composition of embodiment 11, wherein the pharmaceutical composition is formulated for administration to a mammal by parenteral administration.

Embodiment 13: A method of treating a disease or disorder in a subject in need thereof, comprising administering to the subject a therapeutically effective amount of the crystalline 5-(3,6-dibromo-9H-carbazol-9-yl)-N,N,N-trimethylpentan-1-aminium chloride, or hydrate thereof, of any one of embodiments 1 to 10, or the pharmaceutical composition of embodiment 11 or 12.

Embodiment 14: The method of embodiment 13, wherein the disease or disorder is a fat-related disorder.

Embodiment 15: The method of embodiment 14, wherein the fat-related disorder is obesity, abnormal fat distribution, diabetes, a cardiovascular disease, obstructive sleep apnea, lipoma, cancer, osteoarthritis, an endocrinologic disease, a reproductive disease, a neurological disease, a psychiatric disease, a rheumatological disease, or an orthopedic disease.

Embodiment 16: The method of embodiment 14, wherein the fat-related disorder is cellulite, excess fat in various body areas, lipoma, fatty tumor disease, a disorder associated with fat accumulation, Dercum's disease, lipedema, or hibernoma.

Embodiment 17: The method of embodiment 13, wherein the disease or disorder is fibrosis associated with a second disease or disorder.

Embodiment 18: The method of embodiment 17, wherein the second disease or disorder is pulmonary fibrosis, liver cirrhosis, endomyocardial fibrosis, old myocardial infarction, atrial fibrosis, mediastinal fibrosis, myelofibrosis, retroperitoneal fibrosis, progressive massive fibrosis, nephrogenic systemic fibrosis, Carpal tunnel syndrome, joint fibrosis, Crohn's Disease, Keloid, Scleroderma, arthrofibrosis, Peyronie's disease, Dupuytren's contracture, or adhesive capsulitis.

Embodiment 19: The method of claim 13, wherein the disease or disorder is liposarcoma or a solid tumor.

Embodiment 20: The method of embodiment 13, wherein the disease or disorder is a lipoma.

Embodiment 21: The method of embodiment 13, wherein the disease or disorder is angiolipoma.

Embodiment 22: Use of the crystalline 5-(3,6-dibromo-9H-carbazol-9-yl)-N,N,N-trimethylpentan-1-aminium chloride of any one of embodiments 1 to 10, or hydrate thereof, or the pharmaceutical composition of embodiment 11 or 12, in the manufacture of a medicament for the treatment of a disease or disorder in a subject in need thereof.

Embodiment 23: The use of embodiment 22, wherein the disease or disorder is a fat-related disorder.

Embodiment 24: The use of embodiment 23, wherein the fat-related disorder is obesity, abnormal fat distribution, diabetes, a cardiovascular disease, obstructive sleep apnea, lipoma, cancer, osteoarthritis, an endocrinologic disease, a reproductive disease, a neurological disease, a psychiatric disease, a rheumatological disease, or an orthopedic disease.

Embodiment 25: The use of embodiment 23, wherein the fat-related disorder is cellulite, excess fat in various body areas, lipoma, fatty tumor disease, a disorder associated with fat accumulation, Dercum's disease, lipedema, or hibernoma.

Embodiment 26: The use of embodiment 22, wherein the disease or disorder is fibrosis associated with a second disease or disorder.

Embodiment 27: The use of embodiment 26, wherein the second disease or disorder is pulmonary fibrosis, liver cirrhosis, endomyocardial fibrosis, old myocardial infarction, atrial fibrosis, mediastinal fibrosis, myelofibrosis, retroperitoneal fibrosis, progressive massive fibrosis, nephrogenic systemic fibrosis, Carpal tunnel syndrome, joint fibrosis, Crohn's Disease. Keloid, Scleroderma, arthrofibrosis, Peyronie's disease, Dupuytren's contracture, or adhesive capsulitis.

Embodiment 28: The use of embodiment 22, wherein the disease or disorder is liposarcoma or a solid tumor.

Embodiment 29: The use of embodiment 22, wherein the disease or disorder is angiolipoma.

Embodiment 30: Anhydrous crystalline 5-(3,6-dibromo-9H-carbazol-9-yl)-N,N,N-trimethylpentan-1-aminium chloride (Compound A):

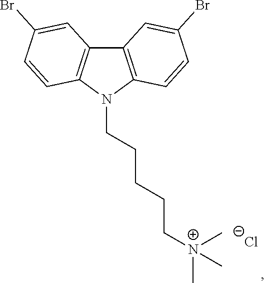 [SMILES: C[N+](C)(C)CCCCCn1c2ccc(Br)cc2c2cc(Br)ccc21 ; [Cl-]]

the anhydrous crystalline 5-(3,6-dibromo-9H-carbazol-9-yl)-N,N,N-trimethylpentan-1-aminium chloride is characterized as having:
   (a) an X-ray powder diffraction pattern comprising peaks at 3.0±0.2° 2-θ, 22.3±0.2° 2-θ, and 31.2±0.2° 2-θ, as measured using Cu Kα1 radiation with an X-ray wavelength of 1.54060 Å;
   (b) an X-ray powder diffraction pattern substantially the same as shown in FIG. 8;
   or
   (c) combinations thereof.

Embodiment 31: The anhydrous crystalline 5-(3,6-dibromo-9H-carbazol-9-yl)-N,N,N-trimethylpentan-1-aminium chloride of embodiment 30, characterized as having an X-ray powder diffraction pattern comprising peaks at 3.0±0.2° 2-θ, 22.3±0.2° 2-θ, and 31.2±0.2° 2-θ, as measured using Cu Kα1 radiation with an X-ray wavelength of 1.54060 Å.

Embodiment 32: The anhydrous crystalline 5-(3,6-dibromo-9H-carbazol-9-yl)-N,N,N-trimethylpentan-1-aminium chloride of embodiment 30 or embodiment 31, wherein the anhydrous crystalline 5-(3,6-dibromo-9H-carbazol-9-yl)-N,N,N-trimethylpentan-1-aminium chloride is characterized as having an X-ray powder diffraction pattern substantially the same as shown in FIG. 8, as measured using Cu Kα1 radiation with an X-ray wavelength of 1.54060 Å.

Embodiment 33: A mixture of anhydrous crystalline forms of 5-(3,6-dibromo-9H-carbazol-9-yl)-N,N,N-trimethylpentan-1-aminium chloride (Compound A):

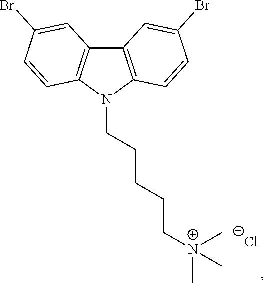 [SMILES: C[N+](C)(C)CCCCCn1c2ccc(Br)cc2c2cc(Br)ccc21 ; [Cl-]]

Embodiment 34: The mixture of embodiment 33, wherein the mixture comprises anhydrous crystalline 5-(3,6-dibromo-9H-carbazol-9-yl)-N,N,N-trimethylpentan-1-aminium chloride is characterized as having an X-ray powder diffraction pattern comprising peaks at 3.0±0.2° 2-θ, 4.8±0.2° 2-θ, 22.3±0.2° 2-θ, 24.8±0.2° 2-θ, 26.5±0.2° 2-θ, and 31.2±0.2° 2-θ, as measured using Cu Kα1 radiation with an X-ray wavelength of 1.54060 Å.

Embodiment 35: The mixture of embodiment 33 or embodiment 34, wherein the anhydrous crystalline forms of Compound A are formed by a process comprising exposing Compound A to an elevated temperature.

Embodiment 36: The mixture of embodiment 35, wherein the elevated temperature is greater than about 90 °C.

Embodiment 37: The mixture of embodiment 35, wherein the elevated temperature is greater than about 100 °C.

Embodiment 38: The mixture of embodiment 35, wherein the elevated temperature is greater than about 150 °C.

Embodiment 39: The mixture of embodiment 35, wherein the elevated temperature is greater than about 200 °C.

Embodiment 40: The mixture of any one of embodiments 33-39, wherein the anhydrous crystalline forms of Compound A are formed by a process comprising drying Compound A to a lower water content.

Embodiment 41: The mixture of embodiment 40, wherein the water content is lower than about 5% (w/w).

Embodiment 42: The mixture of embodiment 40, wherein the water content is lower than about 2.5% (w/w).

Embodiment 43: The mixture of embodiment 40, wherein the water content is lower than about 1% (w/w.)

Embodiment 44: A process for the preparation of anhydrous crystalline 5-(3,6-dibromo-9H-carbazol-9-yl)-N,N,N-trimethylpentan-1-aminium chloride (Compound A), the process comprising reducing a water content in a sample of Compound A and maintaining the sample at a low relative humidity.

Embodiment 45: The process of embodiment 44, wherein reducing the water content comprises heating the sample of Compound A.

Embodiment 46: The process of embodiment 44 or 45, wherein reducing the water content comprises heating the sample of Compound A to a temperature greater than about 90 °C.

Embodiment 47: The process of embodiment 44 or 45, wherein reducing the water content comprises heating the sample of Compound A to a temperature is greater than about 100 °C.

Embodiment 48: The process of embodiment 44 or 45, wherein reducing the water content comprises heating the sample of Compound A to a temperature is greater than about 150 °C.

Embodiment 49: The process of embodiment 44 or 45, wherein reducing the water content comprises heating the sample of Compound A to a temperature is greater than about 200 °C.

Embodiment 50: The process of any one of embodiments 44-49, wherein the low relative humidity is less than about 30%.

Embodiment 51: The process of any one of embodiments 4449, wherein the low relative humidity is less than about 20%.

Embodiment 52: The process of any one of embodiments 44-49, wherein the low relative humidity is less than about 10%.

Embodiment 53: The process of any one of embodiments 44-49, wherein the water content in the sample is reduced to lower than about 5% (w/w).

Embodiment 54: The process of any one of embodiments 4449, wherein the water content in the sample is reduced to lower than about 2.5% (w/w).

Embodiment 55: The process of any one of embodiments 44-49, wherein the water content in the sample is reduced to lower than about 1% (w/w).

Embodiment 56: A process for the preparation of Form 1 of crystalline 5-(3,6-dibromo-9H-carbazol-9-yl)-N,N,N-trimethylpentan-1-aminium chloride (Compound A Form 1), the process comprising exposing a sample of Compound A to a humid environment.

Embodiment 57: The process of embodiment 56, wherein the sample of Compound A is an unstable crystalline form.

Embodiment 58: The process of embodiment 56 or 57, wherein the sample of Compound A is anhydrous.

Embodiment 59: The process of embodiment 58, wherein the sample of Compound A is anhydrous and is characterized as having:
(a) an X-ray powder diffraction pattern comprising peaks at 3.0±0.2° 2-θ, 22.3±0.2° 2-θ, and 31.2±0.2° 2-θ, as measured using Cu Kα1 radiation with an X-ray wavelength of 1.54060 Å;
(b) an X-ray powder diffraction pattern substantially the same as shown in FIG. 8; or
(c) combinations thereof.

Embodiment 60: The process of embodiment 58, wherein the sample of Compound A is anhydrous and is characterized as having:
(a) an X-ray powder diffraction pattern comprising peaks at 4.8±0.2° 2-θ, 26.5±0.2° 2-θ, and 24.8±0.2° 2-θ, as measured using Cu Kα1 radiation with an X-ray wavelength of 1.54060 Å;
(b) an X-ray powder diffraction pattern substantially the same as shown in FIG. 4 as measured using Cu Kα1 radiation with an X-ray wavelength of 1.54060 Å; or
(c) combinations thereof.

Embodiment 61: The process of embodiment 58, wherein the sample of Compound A comprises a mixture of anhydrous crystalline 5-(3,6-dibromo-9H-carbazol-9-yl)-N,N,N-trimethylpentan-1-aminium chloride.

Embodiment 62: The process of embodiment 61, wherein the mixture of anhydrous crystalline 5-(3,6-dibromo-9H-carbazol-9-yl)-N,N,N-trimethylpentan-1-aminium chloride is characterized as having an X-ray powder diffraction pattern comprising peaks at 3.0±0.2° 2-θ, 4.8±0.2° 2-θ, 22.3±0.2° 2-θ, 24.8±0.2° 2-θ, 26.5±0.2° 2-θ, and 31.2±0.2° 2-θ, as measured using Cu Kα1 radiation with an X-ray wavelength of 1.54060 Å.

Embodiment 63: The process of any one of embodiments 56-62, wherein the humid environment comprises greater than about 30% relative humidity.

Embodiment 64: The process of any one of embodiments 56-62, wherein the humid environment comprises greater than about 40% relative humidity.

Embodiment 65: The process of any one of embodiments 56-62, wherein the humid environment comprises greater than about 50% relative humidity.

Embodiment 66: The process of any one of embodiments 56-62, wherein the humid environment comprises greater than about 60% relative humidity.

Embodiment 67: The process of any one of embodiments 56-62, wherein the humid environment comprises greater than about 70% relative humidity.

Embodiment 68: The process of any one of embodiments 56-62, wherein the humid environment comprises a relative humidity of about 30% to about 70%.

Embodiment 69: The process of any one of embodiments 56-68, wherein the humid environment further comprises a temperature from about 15 °C to about 50 °C.

Embodiment 70: The process of any one of embodiments 56-68, wherein the humid environment further comprises a temperature from about 15 °C to about 40 °C.

Embodiment 71: The process of any one of embodiments 56-68, wherein the humid environment further comprises a temperature from about 20 °C to about 40 °C.

What is claimed is:

1. Anhydrous crystalline 5-(3,6-dibromo-9H-carbazol-9-yl)-N,N,N-trimethylpentan-1-aminium chloride (Compound A): 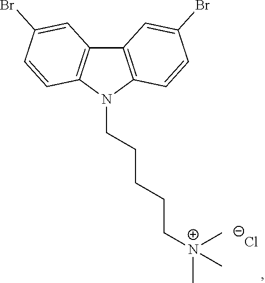 [SMILES: C[N+](C)(C)CCCCCn1c2ccc(Br)cc2c2cc(Br)ccc21 ; [Cl-]]
the anhydrous crystalline 5-(3,6-dibromo-9H-carbazol-9-yl)-N,N,N-trimethylpentan-1-aminium chloride is characterized as having:
(a) an X-ray powder diffraction pattern comprising peaks at 3.0±0.2° 2-θ, 22.3±0.2° 2-θ, and 31.2±0.2° 2-θ, as measured using Cu Kα1 radiation with an X-ray wavelength of 1.54060 Å;
(b) an X-ray powder diffraction pattern substantially the same as shown in FIG. 8;
or
(c) combinations thereof.

2. The anhydrous crystalline 5-(3,6-dibromo-9H-carbazol-9-yl)-N,N,N-trimethylpentan-1-aminium chloride of claim 1, characterized as having an X-ray powder diffraction pattern comprising peaks at 3.0±0.2° 2-θ, 22.3±0.2° 2-θ, and 31.2±0.2° 2-θ, as measured using Cu Kα1 radiation with an X-ray wavelength of 1.54060 Å.

3. The anhydrous crystalline 5-(3,6-dibromo-9H-carbazol-9-yl)-N,N,N-trimethylpentan-1-aminium chloride of claim 1, wherein the anhydrous crystalline 5-(3,6-dibromo-9H-carbazol-9-yl)-N,N,N-trimethylpentan-1-aminium chloride is characterized as having an X-ray powder diffraction pattern substantially the same as shown in FIG. 8, as measured using Cu Kα1 radiation with an X-ray wavelength of 1.54060 Å.

4. A mixture of anhydrous crystalline forms of 5-(3,6-dibromo-9H-carbazol-9-yl)-N,N,N-trimethylpentan-1-aminium chloride (Compound A):

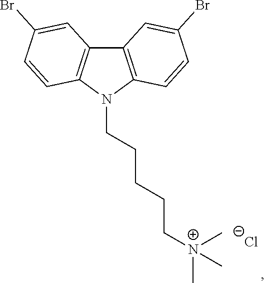 [SMILES: C[N+](C)(C)CCCCCn1c2ccc(Br)cc2c2cc(Br)ccc21 ; [Cl-]]

5. The mixture of claim 4, wherein the mixture comprises anhydrous crystalline 5-(3,6-dibromo-9H-carbazol-9-yl)-N,N,N-trimethylpentan-1-aminium chloride is characterized as having an X-ray powder diffraction pattern comprising peaks at 3.0±0.2° 2-θ, 4.8±0.2° 2-θ, 22.3±0.2° 2-θ, 24.8±0.2° 2-θ, 26.5±0.2° 2-θ, and 31.2±0.2° 2-θ, as measured using Cu Kα1 radiation with an X-ray wavelength of 1.54060 Å.

6. The mixture of claim 4, wherein the anhydrous crystalline forms of Compound A are formed by a process comprising exposing Compound A to an elevated temperature.

7. The mixture of claim 6, wherein the elevated temperature is greater than about 90 °C.

8. The mixture of claim 6, wherein the elevated temperature is greater than about 100 °C.

9. The mixture of claim 6, wherein the elevated temperature is greater than about 150 °C.

10. The mixture of claim 6, wherein the elevated temperature is greater than about 200 °C.

11. The mixture of claim 4, wherein the anhydrous crystalline forms of Compound A are formed by a process comprising drying Compound A to a lower water content.

12. The mixture of claim 11, wherein the water content is lower than about 5% (w/w).

13. The mixture of claim 11, wherein the water content is lower than about 2.5% (w/w).

14. The mixture of claim 11, wherein the water content is lower than about 1% (w/w.)

15. A process for the preparation of anhydrous crystalline 5-(3,6-dibromo-9H-carbazol-9-yl)-N,N,N-trimethylpentan-1-aminium chloride (Compound A), the process comprising reducing a water content in a sample of Compound A and maintaining the sample at a low relative humidity.

16. The process of claim 15, wherein reducing the water content comprises heating the sample of Compound A.

17. The process of claim 15, wherein reducing the water content comprises heating the sample of Compound A to a temperature greater than about 90 °C.

18. The process of claim 15, wherein reducing the water content comprises heating the sample of Compound A to a temperature is greater than about 100 °C.

19. The process of claim 15, wherein reducing the water content comprises heating the sample of Compound A to a temperature is greater than about 150 °C.

20. The process of claim 15, wherein reducing the water content comprises heating the sample of Compound A to a temperature is greater than about 200 °C.

21. The process of claim 15, wherein the low relative humidity is less than about 30%.

22. The process of claim 15, wherein the low relative humidity is less than about 20%.

23. The process of claim 15, wherein the low relative humidity is less than about 10%.

24. The process of claim 15, wherein the water content in the sample is reduced to lower than about 5% (w/w).

25. The process of claim 15, wherein the water content in the sample is reduced to lower than about 2.5% (w/w).

26. The process of claim 15, wherein the water content in the sample is reduced to lower than about 1% (w/w).

27. A process for the preparation of Form 1 of crystalline 5-(3,6-dibromo-9H-carbazol-9-yl)-N,N,N-trimethylpentan-1-aminium chloride (Compound A Form 1), the process comprising exposing a sample of Compound A to a humid environment.

28. The process of claim 27, wherein the sample of Compound A is an unstable crystalline form.

29. The process of claim 27, wherein the sample of Compound A is anhydrous.

30. The process of claim 29, wherein the sample of Compound A is anhydrous and is characterized as having:
(a) an X-ray powder diffraction pattern comprising peaks at 3.0±0.2° 2-θ, 22.3±0.2° 2-θ, and 31.2±0.2° 2-θ, as measured using Cu Kα1 radiation with an X-ray wavelength of 1.54060 Å;
(b) an X-ray powder diffraction pattern substantially the same as shown in FIG. 8; or
(c) combinations thereof.

31. The process of claim 29, wherein the sample of Compound A is anhydrous and is characterized as having:
(a) an X-ray powder diffraction pattern comprising peaks at 4.8±0.2° 2-θ, 26.5±0.2° 2-θ, and 24.8±0.2° 2-θ, as measured using Cu Kα1 radiation with an X-ray wavelength of 1.54060 Å;
(b) an X-ray powder diffraction pattern substantially the same as shown in FIG. 4 as measured using Cu Kα1 radiation with an X-ray wavelength of 1.54060 Å; or
(c) combinations thereof.

32. The process of claim 29, wherein the sample of Compound A comprises a mixture of anhydrous crystalline 5-(3,6-dibromo-9H-carbazol-9-yl)-N,N,N-trimethylpentan-1-aminium chloride.

33. The process of claim 32, wherein the mixture of anhydrous crystalline 5-(3,6-dibromo-9H-carbazol-9-yl)-N,N,N-trimethylpentan-1-aminium chloride is characterized as having an X-ray powder diffraction pattern comprising peaks at 3.0±0.2° 2-θ, 4.8±0.2° 2-θ, 22.3±0.2° 2-θ, 24.8±0.2° 2-θ, 26.5±0.2° 2-θ, and 31.2±0.2° 2-θ, as measured using Cu Kα1 radiation with an X-ray wavelength of 1.54060 Å.

34. The process of claim 27, wherein the humid environment comprises greater than about 30% relative humidity.

35. The process of claim 27, wherein the humid environment comprises greater than about 40% relative humidity.

36. The process of claim 27, wherein the humid environment comprises greater than about 50% relative humidity.

37. The process of claim 27, wherein the humid environment comprises greater than about 60% relative humidity.

38. The process of claim 27, wherein the humid environment comprises greater than about 70% relative humidity.

39. The process of claim 27, wherein the humid environment comprises a relative humidity of about 30% to about 70%.

40. The process of claim 27, wherein the humid environment further comprises a temperature from about 15 °C to about 50 °C.

41. The process of claim 27, wherein the humid environment further comprises a temperature from about 15 °C to about 40 °C.

42. The process of claim 27, wherein the humid environment further comprises a temperature from about 20 °C to about 40 °C.

43. A kit comprising crystalline 5-(3,6-dibromo-9H-carbazol-9-yl)-N,N,N-trimethylpentan-1-aminium chloride (Compound A) and one or more pharmaceutically acceptable excipients.

44. The kit of claim 43, wherein the crystalline 5-(3,6-dibromo-9H-carbazol-9-yl)-N,N,N-trimethylpentan-1-aminium chloride (Compound A) is anhydrous.

45. The kit of claim 43, wherein the crystalline 5-(3,6-dibromo-9H-carbazol-9-yl)-N,N,N-trimethylpentan-1-aminium chloride (Compound A) is hydrated.

46. The kit of any one of claims 43-45, for use in the treatment of a disease or disorder.

47. The kit of claim 46, wherein the disease or disorder is a fat-related disease or disorder, a fibrosis related disease or disorder, a liposarcoma, a solid tumor, or an angiolipoma.
